(19) United States
(12) Patent Application Publication
(10) Pub. No.: US 2009/0304650 A1
(43) Pub. Date: Dec. 10, 2009

(54) Repair of the Bone Marrow Vasculature

(76) Inventors: Edward W. Scott, Gainsville, FL (US); William B. Slayton, Gainesville, FL (US)

Correspondence Address: AKERMAN SENTERFITT, P.O. BOX 3188, WEST PALM BEACH, FL, 33402-3188, US

(21) Appl. No.: 11/914,007

(22) PCT Filed: May 11, 2006
(86) PCT No.: PCT/US06/18159

§ 371 Date: Apr. 7, 2008

Related U.S. Application Data
(60) Provisional application No. 60/679,902, filed on May 11, 2005.

(51) Int. Cl. A61K 35/12; C12N 5/06

(52) U.S. Cl. 424/93.21; 435/325

(57) ABSTRACT

Stem cells repair the marrow vascular niche following bone marrow transplantation. Donor-derived vasculogenesis occurred whether whole bone marrow cells, isolated stem cells, or single stem cells were transplanted. Damaged marrow sinusoids led to hypoxia, followed by upregulation of angiogenic factors hypoxia inducible factor-1 and stromal derived factor-1.

### FIELD OF THE INVENTION

The invention relates to the use of stem cells in the repair of the bone marrow vasculature.

### BACKGROUND

Little is known about blood vessel damage and repair during hematopoietic stem cell transplant. Marrow sinusoidal vessels are “remodeled” following radiation or chemotherapy exposure, suggesting a process of damage and repair. Vascular changes include ballooning of the sinusoids, loss of endothelial integrity, and intramedullary hemorrhage. This has led to speculation that swelling of tissue surrounding the blood vessels causes decreased blood flow in the bone marrow leading to hypoxia, a physiologic trigger for vasculogenesis.

Hypoxia-inducible factor-1 (HIF-1) is a mammalian transcription factor that is expressed in response to hypoxia (Wang et al. 1995. Proc. Natl. Acad. Sci. USA 92: 5510-5514). HIF-1 transactivates genes encoding several glucose transporters and glycolytic enzymes, as well as genes increasing tissue perfusion such as vascular endothelial growth factor (VEGF), inducible nitric oxide synthase, and erythropoietin (Semenza, G. 1999. Annual Review Cell and Development Biology 15: 551-78.). HIF-1 is a heterodimeric molecule composed of a labile alpha (HIF-1α) and a constitutive beta (HIF-1β/ARNT aryl hydrocarbon nuclear transporter) subunit. In normoxia (normal oxygen tension), HIF-1α protein is rapidly degraded via ubiquitination and proteasomal digestion. In contrast HIF-1β is stable and equivalently expressed in normoxia and hypoxia. Thus the major regulation of the transcriptional activity of HIF-1 is due to the HIF-1α component.

Structural analysis of HIF-1α has indicated that dimerization requires two domains that have been termed HLH and PAS, while DNA binding is mediated by a basic domain (Semenza et al. 1997. Kid. Int. 51: 553-555). Further, two transactivation domains have been identified in the C-terminal half of HIF-1α (Jiang, et al. 1997. J. Biol. Chem. 272: 19253-19260).

HIF-1α degradation is mediated by an approximately 200-amino acid domain that has been termed the “oxygen-dependent degradation domain” (ODD) (Huang, L., J. Gu, M. Schau, and H. Bunn. 1998. Proc. Natl. Acad. Sci. U.S.A. 95: 7987-92). Cells transfected with cDNA encoding HIF-1α in which the ODD is deleted (HIF-1αΔODD) demonstrate constitutively active HIF-1α protein regardless of oxygen tension (Huang, L., J. Gu, M. Schau, and H. Bunn. 1998. Proc. Natl. Acad. Sci. U.S.A. 95: 7987-92). A number of stable forms of HIF-1α with deletions in the ODD are described in U.S. Pat. No. 6,124,131.

HIF-1α is required for both embryonic development (Ryan, H., J. Lo, and R. Johnson. 1998. EMBO Journal 17: 3005-15) (Iyer, N. et al. 1998. Genes and Development 12: 149-62) and growth of tumor explants (Ryan, H., J. Lo, and R. Johnson. 1998. EMBO Journal 17: 3005-15). In adult animals HIF-1α is overexpressed in epithelial cancers and high-grade pre-malignant lesions (Zhong, H., et al. 1998. Cancer Research 58: 5280-5284), ischemic cardiac muscle (Lee, S., et al. 2000b. New England Journal of Medicine 342: 626-633), and healing wounds (Elson, D. et al. 2000. Cancer Research 60: 6189-6195).

Successful repair of the vascular niche may be a critical step to restoring hematopoiesis following bone marrow transplant and recovery from chemotherapy. There is therefore, an urgent need in the art to provide a safe, efficient and fast repair of the vasculature following stem cell transplant and to promote wound healing.

### SUMMARY

Donor-derived hematopoietic stem cells of the invention repair the bone marrow vascular niche following stem cell transplant. Sinusoidal injury following irradiation is extensive, and reconstitution of the vascular niche is one of the first steps to successful hematopoietic engraftment. Repair of the bone marrow vascular niche is one of the primary roles of the hematopoietic stem cell following stem cell transplant.

In a preferred embodiment, a composition comprises a stem cell derived from a mammalian donor. The stem cell preferably expresses an angiogenic factor such as hypoxia inducible factor-1. Expression of an angiogenic factor is preferably by a vector that encodes for the angiogenic factor. In accordance with the invention, the angiogenic factor is secreted. Optionally, the stem cell can be cultured in a pharmaceutical composition which modulates the nitric oxide pathway and/or the pharmaceutical composition is administered to a patient prior to, in conjunction with and/or post administration of the stem cell.

In one aspect of the invention, the vector is a plasmid, virus vector and can be inducibly regulated.

In another preferred embodiment, the stem cell is derived from bone marrow. In accordance with the invention, the stem cell is preferably, Lin⁻; Sca-1⁺; c-kit⁺ and/or combinations thereof.

In another preferred embodiment, the stem cell is Lin⁻ Sca-1⁺ c-kit⁺.

In another preferred embodiment, the stem cell is cultured with a compound which modulates nitric oxide pathways. Examples of compounds that regulate the nitric oxide pathway include, but not limited to: sildenafil (Viagra®), vardenafil (Levitra®), tadalafil (Clalis®), apolipoprotein-E, nitroglycerine, L-arginine, nitrate esters, isoamylynitrite, SIN-1, cysteine, dithiothreitol, N-acetylcysteine, mercaptosuccinic acid, thiosalicylic acid, and methylthiosalicylic acid.

In another preferred embodiment, the stem cell is transduced with a vector expressing pro-angiogenic growth factors. Examples of pro-angiogenic factors include, but are not limited to: SDF-1, HGF, IL-8, PGF, TGF and TNF. Preferably, the pro-angiogenic factors are secreted extracellularly.

In another preferred embodiment, the invention provides an isolated or purified population of mammalian hematopoietic stem cells comprising at least one stem cell marker and wherein the purified population of mammalian hematopoietic stem cells are substantially free of cells that do not express the stem cell marker. Preferably, the isolated or purified population of mammalian hematopoietic stem cells are Lin⁻ Sca⁺ and are derived from postnatal mammals. The stem cells can also be Lin⁻ Sca⁺ CD34⁺.

In another preferred embodiment, the isolated or purified population of mammalian hematopoietic stem cells are treated with compounds that modulate the nitric oxide production pathway. Examples of compounds that modulate the nitric oxide pathway include but are not limited to: sildenafil (Viagra®), vardenafil (Levitra®), tadalafil (Clalis®), apolipoprotein-E, nitroglycerine, L-argenine, nitrate esters, isoamylynitrite, SIN-1, cysteine, dithiothreitol, N-acetylcysteine, mercaptosuccinic acid, thiosalicylic acid, and methylthiosalicylic acid.

In another preferred embodiment, the isolated or purified population of mammalian hematopoietic stem cells that are Lin⁻ Sca⁺ c-kit⁺ constitute 15-100% of the total population.

In one preferred embodiment, the isolated or purified population of mammalian hematopoietic stem cells of claim 19, wherein the purified population of mammalian endothelial stem cells that are Lin⁻ Sca⁺ CD34⁺ constitute 15-100% of the total population.

In another preferred embodiment, the invention provides a method of reconstituting hematopoiesis and/or repair of vasculature in a patient comprising administering to a patient a stem cell identified by marker phenotype of Lin⁻ Sca⁺ c-kit⁺. Preferably, at least one Lin⁻ Sca⁺ c-kit⁺ up to about 2×10⁶ Lin⁻ Sca⁺ c-kit⁺ stem cells are administered to a patient.

In accordance with the invention, the stem cells can be administered with one or more compounds that modulate the nitric oxide pathway. In one aspect of the invention, the compounds that modulate the nitric oxide pathway are administered prior to, in conjunction with, and/or post administration of the stem cells.

In another preferred embodiment, a kit is provided comprising stem cell identified by marker phenotype of Lin⁻ Sca⁺ c-kit⁺.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIGS. 1A to 1D are histochemical stains showing the effects of irradiation on bone marrow. FIG. 1A shows a femoral section from a healthy C57 BL6 control animals stained with hematoxylin and eosin (200×). FIG. 1B shows a femoral section one week after 950cGy gamma irradiation. FIG. 1C shows the results of immunohistochemical staining for caspase-3. Caspase-3 signal from femoral section from mouse that received 950 cGy gamma irradiation, 7 days post-transplant. FIG. 1D shows the caspase-3 expression in a C57/BL6 control. (400×).

FIGS. 2A to 2I are photographs of stains showing engraftrnent occurs along the scaffold of dying blood vessels. FIG. 2A shows the results using IgG as a control, one week post-transplant. FIG. 2B shows the results from one week post-transplant of GFP positive whole bone marrow. Section is stained with anti-GFP antibody followed by a secondary antibody conjugated to Alexafluor 488. FIG. 2C shows a cluster of GFPP^{pos} donor-derived cells (green) within a blood vessel day 5 post-transplant. This cluster is next to the endosteal surface, stained with CD31 antibody followed by secondary antibody conjugated to Alexaflour 594 (red, white arrow). FIG. 2D is a photograph showing the results using different stains. The following sections were stained with the FISH probe for X-chromosome (Cy-3, red), and Y-chromosome (FITC, green) Femoral bone marrow, 8-days post sex mismatched transplant (male into female). FISH probe for Y-chromosome is green (FITC) and X-chromosome is red (Cy-3). Donor derived cells form tubular structures (white arrows) along the endosteal surface of the bone (arrow heads). FIG. 2E shows a blood vessel developing along endosteal (white arrows) surface one week post-transplant (blue=Dapi). Each nucleus contains one green and one red signal, making fusion an unlikely explanation for this activity. FIG. 2F is a photograph showing donor derived endothelial cells (arrow) meeting host endothelium (arrow head) in the middle of the marrow cavity, 8 days post-transplant. FIG. 2G is a photograph showing donor derived cells migrating along regressing vessels 8 days post transplant. FIG. 2H is a photograph showing donor-derived cells then filling in the space between the regressing vessels one month post-transplant of 2000 SKL cells. FIG. 2I shows a large vessel cross section one month post transplant of 2000 SKL cells. All of the cells lining the vessel are male and therefore donor-derived.

FIGS. 3A to 3I are photographs (FIGS. 3A-3H) and graph (FIG. 3I) showing that cell lining tubes are functional endothelium. FIG. 3A is a photograph of histochemically stained cells showing that male cells that are CD31^{pos} (red) lining develop sinusoid 1 week post-transplant. FIG. 3B is a histochemical stain showing CD31 and GFP positive endothelial cell lining sinusoids in a mouse receiving 2000 SKL cells, one month post-transplant. FIG. 3C is a histochemical stain showing that male cells also express von Willebrand factor, one week post-transplant (red CD31^{pos}). FIG. 3D is a histochemical stain showing GFP^{pos} cells in a secondary transplant recipient that express CD31 (red), three months post-transplant. FIG. 3E is a histochemical stain showing that sinusoidal blood vessels have endocytosed DiI Ac-LDL uptake (red) in a healthy mouse bone marrow 4 hours after injection. FIG. 3F is a histochemical stain showing transplanted mouse 4 months post-transplant with GFP positive Ac-LDL positive cell lining the blood vessel. FIG. 3G is a histochemical stain showing GFP^{pos} cell lining sinusoid that has endocytosed AcLDL. FIG. 3H is a histochemical stain showing the results from ploidy analysis from lymphocytes from a lymph node of a healthy animal cells that are predominantly 2N. FIG. 3I is a FACS scan showing sorted CD31^{pos} CD45^{low/neg} GFP^{pos} cells are similar to the lymph node control.

FIGS. 4A to 4D show the results obtained from a single cell transplant. Irradiated animals received a single SKL cells from a GFP donor as well as 100,000 Sca-1 depleted C57/BL6 bone marrow cells 7-months prior to analysis. FIG. 4A shows a FACS scan of peripheral blood engraftment of T-cells, B-cells, myeloid cells and platelets. FIG. 4B is a FACS scan showing the endothelial subset based on CD45 and CD31 expression. R4 contains endothelial cells. Arrows point to histograms showing the green fluorescence of cells in R4 from C57/B6 controls (above) and an animal nine months post-transplant of a single GFP^{pos} SKL cell FIG. 4C is a histochemical stain showing GFP positive endothelial cells in recipients of a single cell transplant. FIG. 4D is a histochemical stain showing staining by IgG control antibodies.

FIGS. 5A to 5D are graphs and FIGS. 5E to 5I are histochemical stains showing lethal irradiation leads to hypoxia within the bone marrow, upregulation of hypoxia inducible factor-1, and upregulation of stromal derived factor-1. FIG. 5A is a graph showing the percentage of cells binding pimonidazole. 30 minutes prior to sacrifice, animals were injected with pimonidazole. Flushed bone marrow was treated with anti-pimonidazole antibody conjugated to FITC. Hypoxic cells were detected by flow cytometry at each time point during the first week post-irradiation. FIG. 5B is a graph showing the results from densitometric scanning of m-RNA expression of hypoxia inducible factor-1α detected by RT-PCR. FIG. 5C is a graph showing the results from densitometric scanning of stromal derived factor-1 assayed by PCR. Hypoxanthine-guanine ribosyl transferase (HPRT) m-RNA was assayed as an internal control. FIG. 5D is a graph showing SDF-1 protein expression assayed by ELISA. (Bar chart data for HIF-1 and SDF-1 were combined with duplicated assays from three separate animals). FIG. 5E shows results from immunohistochemistry for SDF-1 (red) and HIF-1 (green) in a healthy C57/BL6 control bone marrow. FIG. 5F is an immunohistochemical stain showing results obtained three days post-irradiation. Both HIF-1 and SDF-1 are upregulated along the endosteal surface. FIG. 5G is an immunohistochemical stain showing HIF-1 and SDF-1 were most apparent along the endosteal surface and within the bone. FIG. 5H is an immunohistochemical stain showing a vessel penetrating through bone that expresses both HIF-1 and SDF-1. FIG. 5I is an immunohistochemical stain showing HIF-1 expression proceeds inward 5 days post-irradiation.

FIGS. 6A to 6D is a schematic illustration of a model for engraftment and endothelial repair. FIG. 6A represents healthy bone marrow. Hematopoietic and endothelial cells are host derived (blue). FIG. 6B represents 3 days post-irradiation. Hypoxia leads to upregulation of HIF-1 and SDF-1 along the endosteal surface and the endosteal blood vessels. FIG. 6C represents 5 days post-irradiation. SDF-1 is expressed along vessels near the center of the bone marrow. Progenitor cells migrate along SDF-1 gradients. FIG. 6D represents 14 days post transplant. Engrafting cells differentiate into both endothelium and hematopoietic cells as they repopulate the bone marrow. The sinusoidal vasculature is remodeled based on the migratory patterns of the engrafting HSCs, EPCs, and HPCs.

FIGS. 7A and 7B is a scan of a cyto stain showing sections from a female mouse transplanted with male bone marrow. The sections are of the bone marrow after transplant. The dark brown staining is for an endothelial specific marker—MECA 32 that stains new endothelium. The green dots are Y chromosome showing male cells, the red dots are the X chromosome. Essentially all of the new sinusoids are being made by male Y positive cells in this area of the bone. This shows support that donor HSC stem cells are first required to rebuild the vascular niche before a successful engraftment can occur. Thus adult hemangioblast activity can be used to improve bone marrow transplant engraftment rates and outcome.

### DETAILED DESCRIPTION

Successful repair of the vascular niche may be a critical step to restoring hematopoiesis following bone marrow transplant and recovery from chemotherapy. The invention provides compositions and methods for the repair of the vascular niche following stem cell transplant. In particular, the compositions repair widespread endothelial damage following irradiation and repair the vascular niche by donor-derived cells.

### DEFINITIONS

The present section provides definitions of the terms used in the present invention in order to facilitate a better understanding of the invention.

As used herein, the singular forms “a”, “an” and “the” include plural referents unless the context clearly dictates otherwise.

A “stem cell” is a relatively undifferentiated cell that can be induced to proliferate and that can produce progeny that subsequently differentiate into one or more mature cell types, while also retaining one or more cells with parental developmental potential. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype.

“Progenitor cells” have a cellular phenotype that is more primitive (i.e., is at an earlier step along a developmental pathway or progression than is a fully differentiated cell). Often, progenitor cells also have significant or very high proliferative potential. Progenitor cells may give rise to multiple distinct differentiated cell types or to a single differentiated cell type, depending on the developmental pathway and on the environment in which the cells develop and differentiate. Like stem cells, it is possible that cells that begin as progenitor cells might proceed toward a differentiated phenotype, but then “reverse” and re-express the progenitor cell phenotype.

“Differentiation” refers to the developmental process whereby cells assume a specialized phenotype, i.e., acquire one or more characteristics or functions distinct from other cell types. In most uses, the differentiated phenotype refers to a cell phenotype that is at the mature endpoint in some developmental pathway. In many but not all tissues, the process of differentiation is coupled with exit from the cell cycle—in these cases, the cells lose or greatly restrict their capacity to proliferate when they differentiate.

“Subatmospheric” conditions mean any oxygen concentration below about 20%, preferably below about 15%, more preferably below about 10%, at sea level. The term subatmoshpheric may be used herein interchangeably with “low oxygen conditions” defined above.

“Atmospheric O₂ conditions” are those conditions found in the air, i.e., 20-21% O₂. As used herein this term is used interchangeably with the term “traditional” O₂ conditions as traditional tissue culture incubators are kept at atmospheric O₂ conditions.

“Physiologic” oxygen levels are the range of oxygen levels normally found in healthy tissues and organs. These levels vary depending on tissue type. However, it is of note that this rate is below 15% in all tissues and below 8% in most tissues. Thus the physiological oxygen levels can range from about 15% to about 1.5% depending upon the region of the body being measured.

“Hypoxia” occurs when the normal physiologic levels of oxygen are not supplied to a cell or tissue. “Normoxia” refers to normal physiologic levels of oxygen for the particular cell type, cell state or tissue in question. “Anoxia” is the absence of oxygen. “Hypoxic conditions” are those leading to cellular hypoxia. These conditions depend on cell type, and on the specific architecture or position of a cell within a tissue or organ, as well as the metabolic status of the cell. A critical point is that in most cell biology research of the past 25 years, ambient atmospheric oxygen levels of 20-21% are routinely called and experimentally taken to be “normoxic,” but this assumption is physiologically erroneous. In this historic context, much cell culture literature refers to any condition with oxygen lower than ambient atmospheric as “hypoxic,” but this usage is also physiologically incorrect.

“Acidosis” means that the pH is below normal physiologic levels.

“Enriching” of cells means that the yield (fraction) of cells of one type is increased over the fraction of cells in the starting culture or preparation.

“Proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

“Regeneration” means re-growth of a cell population, organ or tissue after disease or trauma.

The term “subject,” or “patient” as used herein, means a human or non-human animal, including but not limited to mammals such as a dog, cat, horse, cow, pig, sheep, goat, chicken, primate, rat, and mouse.

An “expression vector” is a vector capable of expressing a DNA (or cDNA) molecule cloned into the vector and, in certain cases, producing a polypeptide or protein. Appropriate transcriptional and/or translational control sequences are included in the vector to allow it to be expressed in a cell. Expression of the cloned sequences occurs when the expression vector is introduced into an appropriate host cell. If a eukaryotic expression vector is employed, then the appropriate host cell would be any eukaryotic cell capable of expressing the cloned sequences.

As used herein, the term “administering a molecule to a cell” (e.g., an expression vector, nucleic acid, a angiogenic factor, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

A cell has been “transformed”, “transduced”, or “transfected” by exogenous or heterologous nucleic acids when such nucleic acids have been introduced inside the cell. Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

A “vector” is a composition which can transduce, transfect, transform or infect a cell, thereby causing the cell to express nucleic acids and/or proteins other than those native to the cell, or in a manner not native to the cell. A cell is “transduced” by a nucleic acid when the nucleic acid is translocated into the cell from the extracellular environment. Any method of transferring a nucleic acid into the cell may be used; the term, unless otherwise indicated, does not imply any particular method of delivering a nucleic acid into a cell. A cell is “transformed” by a nucleic acid when the nucleic acid is transduced into the cell and stably replicated. A vector includes a nucleic acid (ordinarily RNA or DNA) to be expressed by the cell. A vector optionally includes materials to aid in achieving entry of the nucleic acid into the cell, such as a viral particle, liposome, protein coating or the like. A “cell transduction vector” is a vector which encodes a nucleic acid capable of stable replication and expression in a cell once the nucleic acid is transduced into the cell.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” or “therapeutic amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference.

### Stem Cell Population

In one preferred embodiment, the population of stem cells is purified. A purified population of stem cells contains a significantly higher proportion of stem cells than the crude population of cells from which the stem cells are isolated. For example, the purification procedure should lead at least to a five fold increase, preferably at least a ten fold increase, more preferably at least a fifteen fold increase, most preferably at least a twenty fold increase, and optimally at least a twenty-five fold increase in stem cells with respect to the total population. The purified population of stem cells should include at least 15%, preferably at least 20%, more preferably at least 25%, most preferably at least 35%, and optimally at least 50% of stem cells.

The purified population of stem cells may be isolated by contacting a crude mixture of cells containing a population of stem cells that express an antigen characteristic of stem cells with a molecule that binds specifically to the extracellular portion of the antigen. Such a technique is known as positive selection.

Procedures used to isolate stem cells are described in detail in the Examples which follow. However, isolation of cells useful in the present invention can be obtained by any method that is well known in the art. For example, bone marrow derived hematopoietic stem cells can be isolated by density gradient centrifugation, e.g., with Ficoll/Hypaque. Specific cell populations can be depleted or enriched using standard methods using stem cell-specific mAbs (e.g., anti-CD34 mabs). Specific cell populations can also be isolated by fluorescence activated cell sorting according to standard methods. Monoclonal antibodies to cell-specific surface markers known in the art and many are commercially available. The binding of the stem cells to the molecule permit the stem cells to be sufficiently distinguished from contaminating cells that do not express the antigen to permit isolating the stem cells from the contaminating cells. For example, Lin⁻, Sca⁺, c-kit⁺, CD34⁺.

The molecule used to separate stem cells from the contaminating cells can be any molecule that binds specifically to the antigen that characterizes the stem cell. The molecule can be, for example, a monoclonal antibody, a fragment of a monoclonal antibody, or, in the case of an antigen that is a receptor, the ligand of that receptor. For example, VEGF. The number of antigens, such as VEGF receptors, characteristic of stem cells found on the surface of such cells, must be sufficient to isolate purified populations of such cells. For example, the number of antigens found on the surface of stem cells should be at least approximately 1,000, preferably at least approximately 5,000, more preferably at least approximately 10,000, most preferably at least approximately 25,000, and optimally at least approximately 100,000. There is no limit as to the number of antigens contained on the surface of the cells. For example, the cells may contain approximately 150,000, 250,000, 500,000, 1,000,000, or even more antigens on the surface.

The source of stem cells may be any natural or non-natural mixture of cells that contains stem cells. The source may be derived from an embryonic mammal, or from the post-natal mammal. One source of cells is the hematopoietic micro-environment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

Either before or after the crude cell populations are purified as described above, the population of stem cells may be further concentrated by methods known in the art. For example, the stem cells can be enriched by positive selection for one or more antigens characteristic of stem cells. Such antigens include, for example, FLK-1, CD34, and AC133. For example, human stem cells may be pre-purified or post-purified by means of an anti-CD34 antibody, such as the anti-My-10 monoclonal antibody described by Civin in U.S. Pat. No. 5,130,144. The hybridoma cell line that expresses the anti-My monoclonal antibody is available from the American Type Culture Collection, 12301 Parklawn Drive, Rockville, Md. 20852, USA. Some additional sources of antibodies capable of selecting CD34+ cells include AMAC, Westbrook, Me.; Coulter, Hialea, Fla.; and Becton Dickinson, Mountain View, Calif. CD34⁺ cells may also be isolated by means of comparable antibodies, which may be produced by methods known in the art, such as those described by Civin in U.S. Pat. No. 5,130,144.

In addition, or as an alternative to, the enrichment with anti-CD34 antibodies, populations of stem cells may also be further enriched with anti-Sca antibodies; with the AC 133 antibodies described by Yin et al., Blood 90, 5002-5112 (1997) and by Miraglia et al., Blood, 90, 50135021 (1997). The AC133 antibodies may be prepared in accordance with Yin et al.; ibid, or purchased from Miltenyi Biotec.

In accordance with the invention, stem cells can also be detected using for example, antibodies to c-kit. The c-kit proto-oncogene encodes a transmembrane tyrosine kinase receptor for an unidentified ligand and is a member of the colony stimulating factor-1 (CSF-1)—platelet-derived growth factor (PDGF)—kit receptor subfamily. c-kit was shown to be allelic with the white-spotting (W) locus of the mouse. Mutations at the W locus affect proliferation and/or migration and differentiation of germ cells, pigment cells and distinct cell populations of the hematopoietic system during development and in adult life. The effects on hematopoiesis are on the erythroid and mast cell lineages as well as on stem cells, resulting in a macrocytic anemia which is lethal for homozygotes of the most severe W alleles, and a complete absence of connective tissue and mucosal mast cells. W mutations exert their effects in a cell autonomous manner, and in agreement with this property, c-kit RNA transcripts were shown to be expressed in targets of W mutations (Nocka, K., Majumder, S., Chabot, B., Ray, P., Cervone, M., Bernstein, A. and Besmer, P. (1989) Genes & Dev. 3, 816-826.). High levels of c-kit RNA transcripts were found in primary bone marrow derived mast cells and mast cell lines. Somewhat lower levels were found in melanocytes and erythroid cell lines. The identification of the ligand for c-kit is of significance and interest because of the pleiotropic effects it might have on the different cell types which express c-kit and which are affected by W mutations in vivo. The demonstration of identity of c-kit with the W locus implies a function for the c-kit receptor system in various aspects of melanogenesis, gametogenesis and hematopoiesis during embryogenesis and in the adult animal.

The ligand of the c-kit receptor, KL, has been identified and characterized, based on the known function of c-kit/W in mast cells (Zsebo, K. M., et al., (1990a) Cell 63, 195-201; Zsebo, K. M., et al., Cell 63, 213-214 (1990B). The c-kit receptor in hematopoiesis KL stimulates the proliferation of bone marrow derived and connective tissue mast cells and in erythropoiesis, in combination with erythropoietin, KL promotes the formation of erythroid bursts (day 7-14 BFU-E). Furthermore, recent in vitro experiments with KL have demonstrated enhancement of the proliferation and differentiation of erythroid, myeloid and lymphoid progenitors when used in combination with erythropoietin, GM-CSF, G-CSF and IL-7 respectively suggesting that there is a role for the c-kit receptor system in progenitors of several hematopoietic cell lineages.

As used herein, c-kit ligand protein and polypeptide encompasses both naturally occurring and recombinant forms, i.e., non-naturally occurring forms of the protein and the polypeptide which are sufficiently identically to naturally occurring c-kit to allow possession of similar biological activity. Examples of such polypeptides includes the polypeptides designated KL-1.4 and S-KL, but are not limited to them. Such protein and polypeptides include derivatives and analogs. In one embodiment of this invention, the purified mammalian protein is a murine protein. In another embodiment of this invention, the purified mammalian protein is a human protein.

Cells may be further enriched for stem cells by removing cells that are Lin⁺. Such a method is known as negative selection. Negative selection may be used either before or after positive selection. Thus, molecules, such as antibodies or fragments of antibodies, that bind to all or any combination of CD1, CD2, CD3, CD4, CD5, CD8, CD10, CD11b, CD13, CD14, CD15, CD16, CD19, CD20, CD24, CD25, CD28, CD29, CD33, CD36, CD38, CD41, CD41a, CD56, CD66b, CD66e, CD69, and glycophorin A may be used to remove the unwanted Lin⁺ cells by the same methods described above for positive selection.

The stem cells isolated and purified as described herein are primary cells. The primary cells may be cultured and passaged. Being passaged refers to the dividing of a cell population into portions in order to allow further expansion of the cell population. The stem cells administered therapeutically to mammals, as described below, may be cells that have been passaged, but are preferably primary cells. The primary cells may be cultured, but they are preferably not passaged.

### Techniques of Isolating Stem Cells

A mixture of cells from a suitable source of hematopoietic stem cells, as described above, is harvested from a mammalian donor by methods known in the art. A suitable source is the bone marrow and/or hematopoietic microenvironment. For example, circulating peripheral blood, preferably mobilized (i.e., recruited) as described below, may be removed from a patient. Alternatively, bone marrow may be obtained from a mammal, such as a human patient, undergoing an autologous transplant.

The mixture of cells obtained are exposed to a molecule that binds specifically to the antigen marker characteristic of stem cells. The binding molecule is preferably an antibody or a fragment of an antibody. Antigen markers have been discussed supra and in detail in the Examples which follow. A convenient antigen marker is a VEGF receptor, more specifically a FLK-1 receptor.

The cells that express the antigen marker bind to the binding molecule. The binding molecule distinguishes the bound cells from unbound cells, permitting separation and isolation. If the bound cells do not internalize the molecule, the molecule may be separated from the cell by methods known in the art. For example, antibodies may be separated from cells by a short exposure to a solution having a low pH, or with a protease such as chymotrypsin.

The molecule used for isolating the purified populations of stem cells is advantageously conjugated with labels that expedite identification and separation. Examples of such labels include magnetic beads; biotin, which may be identified or separated by means of its affinity to avidin or streptavidin; fluorochromes, which may be identified or separated by means of a fluorescence-activated cell sorter (FACS, see below), and the like.

If desired, a large proportion of terminally differentiated cells may be removed by initially using a “relatively crude” separation. For example, magnetic bead separations may be used initially to remove large numbers of lineage committed cells. Desirably, at least about 80%, usually at least 70% of the total cells will be removed. Procedures for separation may include but are not limited to, magnetic separation, using antibody-coated magnetic beads, affinity chromatography, cytotoxic agents joined to a monoclonal antibody or used in conjunction with a monoclonal antibody, including but not limited to, complement and cytotoxins, and “panning” with antibody attached to a solid matrix, e.g., plate, elutriation or any other convenient technique.

Techniques providing accurate separation include but are not limited to, flow cytometry, which can have varying degrees of sophistication, e.g., a plurality of color channels, low angle and obtuse light scattering detecting channels, impedance channels, etc. Any technique may be used for isolation as long as the technique does not unduly harm the stem cells. Many such methods are known in the art.

In one embodiment, the binding molecule is attached to a solid support. Some suitable solid supports include nitrocellulose, agarose beads, polystyrene beads, hollow fiber membranes, magnetic beads, and plastic petri dishes. For example, the binding molecule can be covalently linked to Pharmacia Sepharose 6 MB macro beads. The exact conditions and duration of incubation for the solid phase-linked binding molecules with the crude cell mixture will depend upon several factors specific to the system employed, as is well known in the art.

Cells that are bound to the binding molecule are removed from the cell suspension by physically separating the solid support from the remaining cell suspension. For example, the unbound cells may be eluted or washed away with physiologic buffer after allowing sufficient time for the solid support to bind the stem cells. The bound cells are separated from the solid phase by any appropriate method, depending mainly upon the nature of the solid phase and the binding molecule. For example, bound cells can be eluted from a plastic petri dish by vigorous agitation. Alternatively, bound cells can be eluted by enzymatically “nicking” or digesting an enzyme-sensitive “spacer” sequence between the solid phase and an antibody. Suitable spacer sequences bound to agarose beads are commercially available from, for example, Pharmacia.

The eluted, enriched fraction of cells may then be washed with a buffer by centrifugation and preserved in a viable state at low temperatures for later use according to conventional technology. The cells may also be used immediately, for example by being infused intravenously into a recipient.

In a particularly preferred variation of the method described above, blood is withdrawn directly from the circulating peripheral blood of a donor. The blood is percolated continuously through a column containing the solid phase-linked binding molecule, such as an antibody to Flk-1, Sca-1, or c-kit to capture stem cells. The stem cell-depleted blood is returned immediately to the donor's circulatory system by methods known in the art, such as hemapheresis. The blood is processed in this way until a sufficient number of stem cells binds to the column. The stem cells are then isolated from the column by methods known in the art. This method allows rare peripheral blood stem) cells to be harvested from a very large volume of blood, sparing the donor the expense and pain of harvesting bone marrow and the associated risks of anesthesia, analgesia, blood transfusion, and infection. Other methods for isolating the purified populations of stem cells are also known, such as those described infra. For example, such methods include magnetic separation with antibody-coated magnetic beads, and “panning” with an antibody attached to a solid matrix.

Methods for removing unwanted cells by negative selection are also known. For example, unwanted cells in a starting cell population are labeled by an antibody, or by a cocktail of antibodies, to a cell surface protein characteristic of Lin⁺ cells. The unwanted antibody-labeled cells are removed by methods known in the art. For example, the labeled cells can be immobilized on a column that binds to the antibodies and captures the cells.

Alternatively, the antibody that binds the cell surface proteins can be linked to magnetic colloids for capture of unwanted cells on a column surrounded by a magnetic field. This system is currently available through StemCell Technologies Inc., Vancouver, British Columbia, Canada. The remaining cells that flow through the column for collection are enriched in cells that do not express the cell surface proteins that the tetrameric antibodies were directed against. The antibody cocktail that can be used to deplete unwanted Lin⁺ cells can be custom made to include antibodies against lineage specific markers, such as, for example, CD2, CD3, CD4, CD5, CD8, CD10, CD11b, CD13, CD14, CD15, CD16, CD19, CD20, CD24, CD25, CD28, CD29, CD33, CD36, CD38, CD41, CD56, CD66b, CD66e, CD69, and glycophorin A. The desired cells that lack these markers are not lineage committed, i.e. Lin⁻.

### General Fluorescence Activated Cell Sorting (FACS) Protocol

Methods for the separation of desired stem cells are described in the Examples which follow.

In one embodiment, a labeled binding molecule is bound to the stem cells, and the labeled cells are separated by a mechanical cell sorter that detects the presence of the label. The preferred mechanical cell sorter is a fluorescence activated cell sorter (FACS). FACS machines are commercially available. Generally, the following FACS protocol is suitable for this procedure:

A Coulter Epics Eliter sorter is sterilized by running 70% ethanol through the systems. The lines are flushed with sterile distilled water. Cells are incubated with a primary antibody diluted in Hank's balanced salt solution supplemented with 1% bovine serum albumin (HB) for 60 minutes on ice. The cells are washed with HB and incubated with a secondary antibody labeled with fluorescein isothiocyanate (FITC) for 30 minutes on ice. The secondary label binds to the primary antibody. The sorting parameters, such as baseline fluorescence, are determined with an irrelevant primary antibody. The final cell concentration is usually set at one million cells per ml.

While the cells are being labeled, a sort matrix is determined using fluorescent beads as a means of aligning the instrument. Once the appropriate parameters are determined, the cells are sorted and collected in sterile tubes containing medium supplemented with fetal bovine serum and antibiotics, usually penicillin, streptomycin and/or gentamicin. After sorting, the cells are re-analyzed on the FACS to determine the purity of the sort.

Methods for Complete Replacement of Vascular Niche and/or Repair of the Vasculature.

The invention is further directed to a method for inducing neovascularization, neomyogenesis, and neoneurogenesis in a mammal. The method comprises treating the mammal with an effective amount of a purified population of hematopoietic stem cells. Any one of the three types of stem cells may be used to induce any one of the three types of new mature cells, e.g., neovascularization, neomyogenesis, and neoneurogenesis

In this specification, vascularization refers to the development of new blood vessels in a postnatal mammal from endothelial, muscle, or neural stem cells by any means, such as by vasculogenesis followed by linking of the new blood vessels to existing blood vessels, angiogenesis, or the formation of new blood vessels that form as a result of the ability of endothelial stem cells to bind to existing blood vessels and to grow into new blood vessels.

Bone marrow sinusoids are capillaries that collect maturing blood cells and deliver them to the central circulation. In addition to their role as conduit to the blood stream, the sinusoids produce factors that enhance hematopoietic differentiation. Thus, the sinusoidal endothelial cells may provide a niche for differentiating hematopoietic progenitor cells. The sinusoidal “vascular niche” is particularly important to megakaryocyte development. Hematopoietic progenitors are thought to migrate from the endosteal niche, where hematopoietic stem cells are known to reside next to bone and receive signals from osteoblasts, to the more centrally located vascular niche, where progenitors receive proliferation and maturational signals. Furthermore, endothelium produces soluble factors that enhance the survival and function of human severe combined immunodeficiency (SCID) repopulating cells. Repair of the sinusoidal vascular niche may be necessary for successful engraftment in bone marrow transplant.

The bone marrow, in addition to being the main site of hematopoiesis, is a rich source of endothelial progenitor cells (EPCs). EPCs circulate in response to ischemic injury and incorporate into the blood vessels of damaged tissues. However, the role of the EPC in repairing local vascular damage within the bone marrow is unknown. Revascularization restores function and heals injured tissue. Revascularization occurs by at least three distinct processes. Vasculogenesis involves the development of blood vessels from stem cells or undifferentiated tissue. Vasculogenesis occurs in blood islands of the embryonic yolk sac, and was originally thought to occur primarily in the embryo. Angiogenesis involves the sprouting of new vessels from previously existing vessels. Arteriogenesis involves the maturation of pre-existing collateral vessels that enlarge after blockage or damage to a primary vessel. Little is known about blood vessel damage and repair during hematopoietic stem cell transplant. Marrow sinusoidal vessels are “remodeled” following radiation or chemotherapy exposure, suggesting a process of damage and repair. Vascular changes include ballooning of the sinusoids, loss of endothelial integrity, and intramedullary hemorrhage. This has led to speculation that swelling of tissue surrounding the blood vessels causes decreased blood flow in the bone marrow leading to hypoxia, a physiologic trigger for vasculogenesis. Successful repair of the vascular niche may be a critical step to restoring hematopoiesis following bone marrow transplant and recovery from chemotherapy.

This study demonstrates that in abone marrow transplant, HSCs function as hemangioblasts, and extensively repair the sinusoidal vascular niche. This vasculogenesis begins in the endosteal area and moves centrally along a scaffold of dying blood vessels. These new tubes are among the first recognizable donor-derived structures in the bone marrow one week post-transplant, before recognizable hematopoiesis begins. During this period post-transplant, the spleen is the major source of hematopoiesis in the mouse. Splenic hematopoiesis may be necessary to allow the marrow vascular niche to be repaired. Furthermore, donor-derived endothelial repair was observed in all engrafted animals that received a single HSC, suggesting that bone marrow endothelium can be repaired by the activity of the hematopoietic stem cell.

The role of hypoxia in the process of hematopoietic engraftment was also demonstrated. Vasculogenesis and angiogenesis are processes that are triggered by hypoxia. Mammalian cells respond to hypoxia in part by upregulation of hypoxia inducible factor-1, a transcription factor that regulates expression of a numerous angiogenic and hematopoietic cytokines as well as inflammatory mediators. Increased production of the HIF-1α subunit is one way HIF-1 is regulated following hypoxia. HIF-1-β is produced constitutively within the cell, and these two subunits bind to form HIF-1. In normoxic cells, HIF-1 is ubiquitinated by the von Hippel Lindau protein, and destroyed in the proteosome. In hypoxic tissues, HIF-1 is allowed to accumulate and bind to DNA where it serves as a transcription factor for inducing numerous angiogenic cytokines as well as regulating transcription factors for erytropoiesis. Areas of increased HIF-1 expression correspond directly with the areas where endothelial and hem atopoietic progenitors migrate during the first few days post-transplant. Furthermore, the HIF-1 dependent chemokine, stromal derived factor-1, is upregulated in the same areas, providing a stimulus to direct this migration.

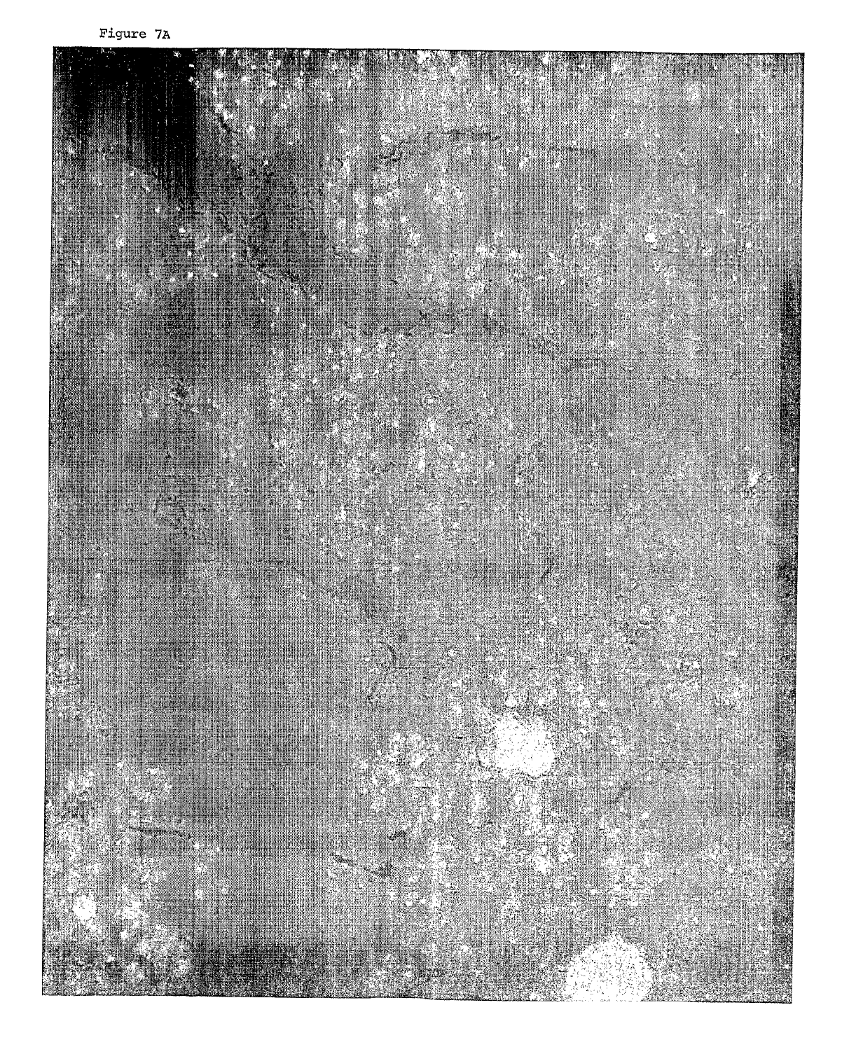

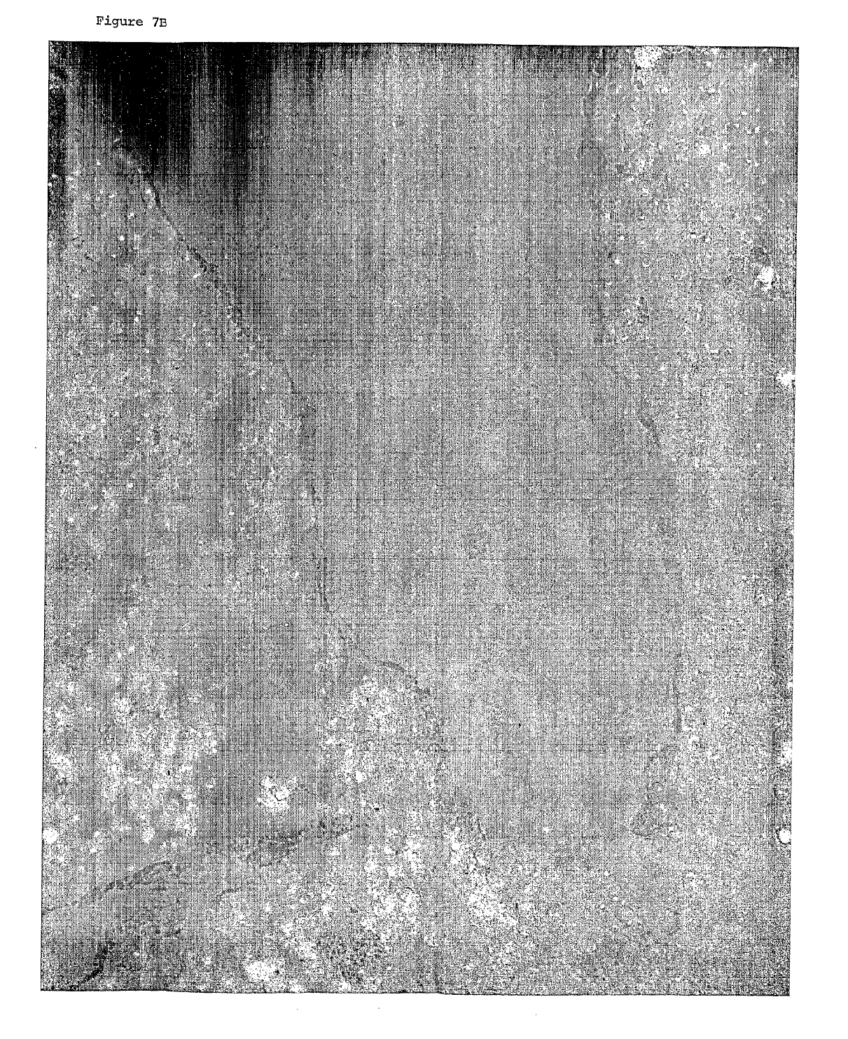

Without wishing to be bound by theory, FIGS. 6A-6D depict a schematic representation of a model of post-transplant vascular repair. FIG. 6A shows a healthy bone marrow, where HSCs inhabit niches that focally express SDF-1. Radiation leads to hypoxia and injury to tissues, first in the endosteal region, and later in regions near the center of the bone. This leads to the production of SDF-1 first in the endosteal region, which results in HSCs, HPCs, and EPCs lining the endosteal surface and the periendosteal blood vessels (FIGS. 6B and 6D). By five days post-radiation, the vessels that branch toward the center of the marrow space from the vessels lining the endosteum are hypoxic, and co-express both HIF-1 and SDF-1. This SDF-1 expression leads HSCs, HPCs, and EPCs inward along these dying blood vessels toward the center of the bone marrow space. FIG. 6D shows how these progenitor cells then produce endothelial cells and hematopoietic cells that fill in the spaces between the newly formed, donor-derived sinusoidal blood vessels. The newly formed vessels do not conform exactly to the vessels they have replaced. This model explains how a wave of HSCs, HPCs, and EPCs might circulate at a time when donor-derived hematopoiesis is beginning. These stem and progenitor cells would be closely associated with the sinusoidal vessels and would have easy access to the blood stream coincident with the recovery of hematopoiesis.

There are numerous conditions that cause the necessity of a mammal to be in need of repair of the vascular “niche.” For example, the mammal may have a wound that requires healing. The wound may be an acute wound, such as those caused by burns and contact with hard and/or sharp objects. For example, patients recovering from surgery, such as cardiovascular surgery, cardiovascular angioplasty, carotid angioplasty, and coronary angioplasty all require neovascularization. The wound may also be a chronic wound. Some examples of chronic wounds include ulcers, such as vascular ulcers and diabetic ulcers.

Inducing vascularization from stem cells is especially effective in increasing cardiac or peripheral (i.e. limb) vascularization. Therefore, the method is especially effective in treating cardiac and peripheral ischemia.

Patients suffering from other conditions also require vascularization. Such conditions include patients having undergone bone marrow transplant, patients suffering from sickle cell anemia and thalassemia.

Mammals in need of neomyogenesis include mammals that suffer from injury to the central nervous system, especially the spinal cord; Parkinson's disease; and Alzheimer's disease. Mammals in need of neoneurogenesis include mammals that suffer from injury to the cardiac or skeletal muscle system, and mammals that suffer from muscular dystrophy.

The purified population of stem cells are introduced into a mammal in any way that will cause the cells to migrate to the site where the stem cells are needed. For example, the stem cells may be introduced into a mammal intravenously, by means of a catheter, or directly into the site by, for example, injection.

The stem cells that are administered to a mammal may be autologous or heterologous. Preferably, the stem cells are autologous to the recipient mammal. For example, the cells may be administered after surgery, preferably approximately 0.1-24 hours after surgery.

The stem cells are recruited into the site that requires new cells and tissues. For example, stem cells may be mobilized (i.e., recruited) into the circulating peripheral blood by means of angiogenic factors such as hypoxia inducible factor-1 (HIF-1); cytokines, such as, for example, G-CSF, GM-CSF, VEGF, SCF (c-kit ligand) and βFGF; chemokines, such as SDF-1, or interleukins, such as interleukins 1 and 8. Stem cells may also be recruited to the circulating peripheral blood of a mammal if the mammal sustains, or is caused to sustain, an injury.

In a preferred embodiment, hypoxia inducible factor-1 and stromal derived factor-1 are up-regulated. The stem cells can be transduced with a vector expressing hypoxia inducible factor 1 and/or administered an effective amount of a compound that modulates the nitric oxide pathway. Examples of such compounds, include, but are not limited to: sildenafil (Viagra®), vardenafil (Levitra®), tadalafil (Clalis®), apolipoprotein-E, nitroglycerine, L-arginine, nitrate esters, isoamylynitrite, SIN-1, cysteine, dithiothreitol, N-acetylcysteine, mercaptosuccinic acid, thiosalicylic acid, and methylthiosalicylic acid.

### Hypoxic Culture Conditions

In another preferred embodiment, stem cells are cultured under hypoxic conditions prior to administration to a patient. By “hypoxic” it is meant an environment with reduced levels of oxygen. Most preferably oxygen levels in cell culture will be 0.1 to 1.0% for the provision of a hypoxic state. Hypoxia may be induced in cells simply by culturing the cells in the presence of lowered oxygen levels. The cells may also be treated with compounds which mimic hypoxia and cause up regulation of HIFα subunit expression. Such compounds include iron chelators, cobalt (II), nickel (II) or manganese (II), all of which may be used at a concentration of 20 to 500 μM. such as 100 μM. Iron chelators include desferrioxamine, O-phenanthroline or hydroxypyridinones (e.g. 1,2-diethyl hydroxypyridinone (CP94) or 1,2-dimethyl hydroxypyridinone (CP20)).

Suitable medium and conditions for generating primary cultures are well known in the art and vary depending on cell type. For example, skeletal muscle, bone, neurons, skin, liver, and embryonic stem cells are all grown in media differing in their specific contents. Furthermore, media for one cell type may differ significantly from lab to lab and institution to institution. As a general principle, when the goal of culturing is to keep cells dividing, serum is added to the medium in relatively large quantities (10-20% by volume). Specific purified growth factors or cocktails of multiple growth factors can also be added or sometimes used in lieu of serum. As a general principle, when the goal of culturing is to reinforce differentiation, serum with its mitogens is generally limited (serum about 1-2% by volume). Specific factors or hormones that promote differentiation and/or promote cell cycle arrest can also be used.

Physiologic oxygen and subatmospheric oxygen conditions can be used at any time during the growth and differentiation of cells in culture, as a critical adjunct to selection of specific cell phenotypes, growth and proliferation of specific cell types, or differentiation of specific cell types. In general, physiologic or low oxygen-level culturing is accompanied by methods that limit acidosis of the cultures, such as addition of strong buffer to medium (such as HEPES), and frequent medium changes and changes in CO₂ concentration.

Cells can be exposed to the low oxygen conditions using a variety of means. Physiologic or low oxygen culturing conditions also can be maintained by using commercially-available chambers which are flushed with a pre-determined gas mixture (e.g., as available from Billups-Rothenberg, San Diego Calif.). As an adjunct, medium can be flushed with the same gas mixture prior to cell feeding. In general, it is not possible to maintain physiologic or low oxygen conditions during cell feeding and passaging using these smaller enclosed units, and so, the time for these manipulations should be minimized as much as possible. Any sealed unit can be used for physiologic oxygen or low oxygen level culturing provided that adequate humidification, temperature, and carbon dioxide are provided.

In addition to oxygen, the other gases for culture typically are about 5% carbon dioxide and the remainder is nitrogen, but optionally may contain varying amounts of nitric oxide (starting as low as 3 ppm), carbon monoxide and other gases, both inert and biologically active. Carbon dioxide concentrations typically range around 5% as noted above, but may vary between 2-10%. Both nitric oxide and carbon monoxide are typically administered in very small amounts (i.e. in the ppm range), determined empirically or from the literature.

The optimal physiologic or low oxygen level conditions for any given cell type or any particular desired outcome will vary. A skilled artisan could determine suitable subatmospheric conditions by generating an oxygen dose response curve, in which carbon dioxide is kept constant, and oxygen levels are varied (with nitrogen as the remaining gas). For example, to determine the optimal ambient oxygen culturing conditions for expansion of a cell, one would establish cultures from an organ system. The initial culture is mixed, consisting of some differentiated cells, cells of other developmental lineages or pathways, as well as CNS cells. After exposure to the various oxygen levels (e.g. 1%, 2%, 5%, 10% and 15%), the number and function of cells is assessed by methods appropriate to the system. In some cases, a constellation of molecular markers is available to rapidly identify the cell population. But in other cases, a single marker coupled with proliferation assays is appropriate, while in other cases proliferation assays alone are appropriate. In some cases all or some of the above assays are coupled with bioassays to follow the differentiation potential of the presumed stem cells. Overall, the precise assays used to determine stem cell and/or progenitor response to oxygen levels are dependent on the nature of the system examined as well as available markers and techniques specific to that system.

The timing of physiologic or low oxygen conditions is also part of the oxygen dose response curve. Some cells may be more or less sensitive to oxygen during isolation or immediately after isolation while some cells may respond only after some time in culture. The timing of physiologic or low oxygen conditions absolutely and in relation to other manipulations of the cultures is part of assessing the optimal oxygen culturing conditions. Furthermore, the mitogenic effects of other gases may be synergistic with physiologic or low oxygen conditions. Different gene regulatory networks may be induced by low/physiologic oxygen culturing during different phases of culture. During expansion of the cells, low oxygen may induce gene expression distinct from that induced by low oxygen during differentiation.

The cells are typically exposed to low oxygen level conditions for a time sufficient to enrich the population of progenitor/stem cells compared to other cell types. Typically this is for 1 or more hours, preferably 3 or more hours, more preferably 6 or more hours, and most preferably 12 or more hours, and may be continuous. The temperature during the culture is typically reflective of core body temperature, or about 37° C., but may vary between about 32° C. and about 40° C. Other important embodiments may simply achieve an increase in cell absolute number or promote the survival of cells.

Following an initial exposure to low or physiologic oxygen culturing conditions, cells can be maintained in these conditions or returned to normal laboratory oxygen conditions, depending on the desired outcome. It is understood that the initial medium for isolating the cells, the medium for proliferation of these cells, and the medium for differentiation of these cells can be the same or different. All can be used in conjunction with low or physiologic oxygen level culturing. The medium can be supplemented with a variety of growth factors, cytokines, serum, etc. Examples of suitable growth factors are basic fibroblast growth factor (bFGF), vascular endothelial growth factor (VEGF), epidermal growth factor (EGF), transforming growth factors (TGFα and TGFβ), platelet derived growth factors (PDGFs), hepatocyte growth factor (HGF), insulin-like growth factor (IGF), insulin, erythropoietin (EPO), and colony stimulating factor (CSF). Examples of suitable hormone medium additives are estrogen, progesterone, testosterone or glucocorticoids such as dexamethasone. Examples of cytokine medium additives are interferons, interleukins, or tumor necrosis factor-α (TNFα). One skilled in the art will test additives and culture components at varied oxygen levels, as the oxygen level may alter cell response to, active lifetime of additives or other features affecting their bioactivity. In addition, the surface on which the cells are grown can be plated with a variety of substrates that contribute to survival, growth and/or differentiation of the cells. These substrates include but are not limited to laminin, poly-L-lysine, poly-D-lysine, polyornithine and fibronectin.

### Vector for Gene Therapy

In another embodiment, the invention is directed to a method for producing a vector useful in gene therapy. The method comprises introducing a gene into a stem cell of the invention. The gene is introduced into the stem cell under the control of suitable regulatory sequences so that the stem cells express the protein encoded by the gene.

In a preferred embodiment, the gene is hypoxia inducible factor-1. As described in detail in the Examples which follow, HIF-1 regulates numerous proteins involved with angiogenesis, including stromal derived factor-1 (SDF-1). SDF-1 regulates the migration, survival and proliferation of hematopoietic stem cells and endothelial progenitors.

In another preferred embodiment, the cells may be modified to express or not to express other proteins which are known to interact with HIF (VHL protein, for example Flongin C and Elongin B proteins in the case of VHL and ARNT protein, in the case of HIF-1α subunit protein). von Hippel-Lindau (VHL) disease is a hereditary cancer syndrome characterized by the development of highly vascular tumors that overproduce hypoxia-inducible mRNAs such as vascular endothelial growth factor (VEGF). Human VHL has been cloned and sources of the gene can be readily identified by those of ordinary skill in the art. Its sequence is available as GenBank accession numbers AF010238 and L15409. Other mammalian VHLs are also available, such as murine VHL accession number U12570) and rat (accession numbers U14746 and S80345). Non-mammalian homologues include the VHL-like protein of C. elegans, accession number F08G12. VHL gene sequences may also be obtained by routine cloning techniques, for example by using all or part of the human VHL gene sequence as a probe to recover and to determine the sequence of the VHL gene in other species.

Other examples of genes useful for introduction into isolated stem cells include those that encode Factor VIII, von Willebrand factor, insulin, tissue plasminogen activator, any of the interleukins, or a growth factor. Some examples of interleukins include IL-1, -2, -3, -4, -5, -6, -7, -8, -9, -10, -11, -12, -13, -14, -15, -16, -17, -18, -19, -20, and -21. Some examples of suitable growth factors include erythropoietin, thrombopoietin, PDGF, G-CSF, GM-CSF, IGF, TGFβ, VEGF, BMP (bone morphogenic protein) and CNTF (ciliary neurotrophic factor).

Genes may be introduced into stem cells by methods known in the art. For example, genes may be introduced into endothelial stem cells, as well as into muscle and neural cells, by methods described, for example, in Mulligan, et al., U.S. Pat. No. 5,674,722. The methods described in Mulligan, et al., U.S. Pat. No. 5,674,722 for preparing vectors useful for introducing genes into cells, and for introducing genes into endothelial cells, are incorporated herein by reference.

Briefly, the gene to be introduced into cells is placed under the control of one or more inducible or uninducible regulatory sequences in a standard expression vector and transfected directly into a stem cell by known methods, including, for example, standard lipid-mediated, calcium phosphate, or electroporation techniques.

Alternatively, the gene can be cloned into vectors derived from viruses such as adenovirus, adeno-associated virus, herpesvirus, retrovirus or lentivirus. Gene expression is controlled by inducible or uninducible regulatory sequences.

By virtue of a selectable marker present in the aforementioned vectors, such as a neomycin or puromycin resistance gene or the genes for green or blue fluorescence proteins (GFP or BFP), cells infected or transfected as described above are screened, isolated and propagated to obtain a stock of virus, or cells harboring the virus, expressing the gene of interest. Stem cells may be isolated as described herein and exposed to viral vectors (i.e. infected) in serum-containing or serum-free ex vivo no culture conditions for hours or days in the presence of growth factors such as SCF, Flt-3 ligand, TPO, IL-1, 3, 6, 11, G-CSF, anti-TGFβ antibodies, and mesodermal factors, to support survival and proliferation of the stem cells.

Infected cells can also be isolated by standard drug selection procedures for neomycin and puromycin or by flow cytometric cell sorting for GFP or BFP expressing cells. The transduced cells may be returned back to the patient as described herein, i.e. intravenous injection, intra-tissue injection, or by means of a catheter.

### Gene Therapy

The invention also includes methods for introducing genes to a mammal at a site to which the stem cells of the invention can be recruited. For example, purified populations of endothelial stem cells can be recruited to sites of angiogenesis. Purified populations of muscle stem cells can be recruited to muscles, especially to the musculature of the cardiovascular system, for example, to the heart and blood vessels. Purified populations of neural stem cells can be recruited to the peripheral and central nervous systems, i.e. to the brain and spinal column.

In one embodiment, the method comprises treating the mammal with stem cells, into which a gene under the control of suitable regulatory sequences has been introduced so that the stem cells express the protein encoded by the gene. Examples of suitable genes are those mentioned in connection with the vectors for gene therapy described above. The genes and vectors can be administered to mammals by known methods, including the methods described above.

Selection of the genes to be introduced into stem cells will depend on the application of the gene therapy. For example, gene therapy with endothelial stem cells may be used to promote angiogenesis, inhibit angiogenesis, or to inhibit the growth of tumors.

Some examples of genes useful for promoting angiogenesis include the genes that encode HIF-1, SDF-1, the VEGFs, the cadherins, the integrins, FGFA, FGFp, FGF4, HGF, TGFα, EGF, angiopoietin-1, B61, IL-8, and angiogenin.

Some examples of genes useful for inhibiting angiogenesis for treatment of tumors include the genes that encode soluble KDR, soluble flt-1, KDR antibodies, TGF-β, lymphotoxin, interferon-γ, platelet factor 4, angiopoietin-2, angiostatin, endostatin, thrombospondin, inducible protein-10, and IL-12.

Some examples of genes useful for treating genetic diseases, for example hemophilia or diabetes, include the genes that encode factor VII/von Willebrand, factor IX, and insulin.

The gene can be delivered at a desired site of vascularization. The site of vascularization may be a natural site or an artificially created site. Natural sites of neovascularization include cardiac and peripheral ischemia, tumors, vascular ulcers and other vascular wounds as described above.

The stem cells transfected with a gene therapy vector may be naturally or artificially recruited to the site where the protein expressed by the gene is desired. Recruiting the vector to the site can be induced artificially by administering a suitable chemokine systemically or at the desired site. A suitable molecule is hypoxia inducible factor-1, a chemokine such as stromal derived factor-1 (SDF-1). The endothelial stem cells may also be recruited to the desired site by means of an interleukin, such as IL-1 or IL-8. Other methods include the administration of compounds that regulate the nitric oxide pathway or induce hypoxia. Regulation of the nitric oxide pathway by compounds include, but not limited to: sildenafil (Viagra®), vardenafil (Levitra®), tadalafil (Clalis®), apolipoprotein-E, nitroglycerine, L-arginine, nitrate esters, isoamylynitrite, SIN-1, cysteine, dithiothreitol, N-acetylcysteine, mercaptosuccinic acid, thiosalicylic acid, and methylthiosalicylic acid. The administration of such compounds may be administered to a patient prior to the administration of stem cells, in conjunction with the stem cells after the administration of stem cells or combinations thereof.

The transfected stem cells that are administered to a mammal for gene therapy may be autologous or heterologous. Preferably, the transfected stem cells are autologous.

Other methods for carrying out gene therapy in mammals have been described in the prior art, for example, in Mulligan, et al., U.S. Pat. No. 5,674,722. The methods described in Mulligan, et al., U.S. Pat. No. 5,674,722 for carrying out gene therapy are incorporated herein by reference.

### Isolating Receptors

Receptors and markers that can serve as antigens for making monoclonal antibodies are known in the art. For example, the FLK-1 receptor and gene can be isolated by methods described by Lemischka, U.S. Pat. No. 5,283,354; Matthews, et al., Proc. Natl. Acad. Sci. U.S.A. 88, 9026 (1991); Terman, et al., WO92/14748 and Terman, et al., Biochem. Bioplys. Res. Commun. 187, 1579 (1992). The AC 133 antigen can be prepared as described by Yin et al. in Blood 90, 5002-5112 (1997).

In order to prepare the antigens against which the antibodies are made, nucleic acid molecules that encode the antigen, such as a VEGF receptor, especially the extracellular portions thereof, may be inserted into known vectors for expression using standard recombinant DNA techniques. Standard recombinant DNA techniques are described in Sambrook et al., “Molecular Cloning,” Second Edition, Cold Spring Harbor Laboratory Press (11987) and by Ausubel et al. (Eds) “Current Protocols in Molecular Biology,” Green Publishing Associates/Wiley-Interscience, New York (1990). The vectors may be circular (i.e. plasmids) or non-circular. Standard vectors are available for cloning and expression in a host.

The host may be prokaryotic or eukaryotic. Prokaryotic hosts are preferably E. coli. Preferred eukaryotic hosts include yeast, insect and mammalian cells. Preferred mammalian cells include, for example, CHO, COS and human cells. The DNA inserted into a host may encode the entire extracellular portion, or a soluble fragment thereof. The extracellular portion of the receptor encoded by the DNA is optionally attached at either, or both, the 5′ end or the 3′ end to additional amino acid sequences. The additional amino acid sequence may be attached to the extracellular region in nature, such as those that represent the leader sequence, the transmembrane region and/or the intracellular region of the antigen.

The additional amino acid sequences may also be sequences not attached to the receptor in nature. Preferably, such additional amino acid sequences serve a particular purpose, such as to improve expression levels, solubility, purification, ability to assay, or immunogenicity. Some suitable additional amino acid sequences include, for example, (a) the FLAG peptide optionally attached at either end of the receptor; (b) the Fc portion of an immunoglobulin (Ig), preferably attached at the C-terminus of the receptor; or (c) the enzyme human placental alkaline phosphatase (AP), (Flanagan and Leder, Cell 53, 185-194 (1990)).

### Source of DNA

In order to produce desired nucleic acid molecules for transducing a stem cell, a source of cells that express the gene product is provided. Suitable fetal (i.e. pre-natal) sources include liver, spleen, kidney, or thymus cells. Suitable post-natal sources include bone marrow, umbilical cord endothelial cells or blood, such as circulating peripheral blood, or umbilical cord blood, etc.

Total RNA is prepared by standard procedures from tissue or cells expressing the desired gene product. The total RNA is used to direct cDNA synthesis. Standard methods for isolating RNA and synthesizing cDNA are provided in standard manuals of molecular biology such as, for example, in Sambrook et al., “Molecular Cloning,” Second Edition, Cold Spring Harbor Laboratory Press (1987) and in Ausubel et al., (Eds), “Current Protocols in Molecular Biology,” Greene Associates/Wiley Interscience, New York (1990).

The cDNA may be amplified by known methods. For example, the cDNA may be used as a template for amplification by polymerase chain reaction (PCR); see Saiki et al., Science, 222, 487 (1988) or Mullis et al., U.S. Pat. No. 4,683,195. The sequences of the oligonucleotide primers for the PCR amplification are derived from the sequences of the gene product, e.g. HIF-1α. The oligonucleotides may be synthesized by methods known in the art. Suitable methods include those described by Caruthers in Science, 230, 281-285 (1985).

In order to isolate the entire protein-coding regions, the upstream PCR oligonucleotide primer is complementary to the sequence at the 5′ end, preferably encompassing the ATG start codon and at least 5-10 nucleotides upstream of the start codon. The downstream PCR oligonucleotide primer is complementary to the sequence at the 3′ end of the desired DNA sequence. The desired DNA sequence preferably encodes the entire extracellular portion of the gene product, e.g. HIF-1, and optionally encodes all or part of the transmembrane region, and/or all or part of the intracellular region, including the stop codon. A mixture of upstream and downstream oligonucleotides are used in the PCR amplification. The conditions are optimized for each particular primer pair according to standard procedures. The PCR product may be analyzed by methods known in the art for cDNA having the correct size, corresponding to the sequence between the primers. Suitable methods include, for example, electrophoresis.

Alternatively, the coding region may be amplified in two or more overlapping fragments. The overlapping fragments are designed to include a restriction site permitting the assembly of the intact cDNA from the fragments.

The vector into which the DNA is spliced may comprise segments of chromosomal, non-chromosomal and synthetic DNA sequences. Some suitable prokaryotic cloning vectors include plasmids from E. coli, such as colel, pCRM, pBR322, pMB9, pUC, pKSM, and RP4. Prokaryotic vectors also include derivatives of phage DNA such as M13 and other filamentous single-stranded DNA phages.

### Expression and Isolation of HIF-1.

DNA encoding HIF-1 are inserted into a suitable expression vector and expressed in a suitable prokaryotic or eukaryotic host. Vectors for expressing proteins in bacteria, especially E. coli, are known. Such vectors include the PATH vectors described by Dieckmann and Tzagoloff in J. Biol. Chem. 260, 1513-1520 (1985). These vectors contain DNA sequences that encode anthranilate synthetase (TrpE) followed by a polylinker at the carboxy terminus. Other expression vector systems are based on β-galactosidase (pEX); lambda P_{L}; maltose binding protein (pMAL); and glutathione S-transferase (pGST)—see Gene, 67, 31 (1988) and Peptide Research, 3, 167 (1990). Vectors useful in yeast are available. A suitable example is the 2μ plasmid. Suitable vectors for use in mammalian cells are also known. Such vectors include well-known derivatives of SV-40, adenovirus, retrovirus-derived DNA sequences and shuttle vectors derived from combination of functional mammalian vectors, such as those described above, and functional plasmids and phage DNA.

Further eukaryotic expression vectors are known in the art, e.g., P. J. Southern and P. Berg, J. Mol. Appl. Genet. 1, 327-341 (1982); S. Subramani et al, Mol. Cell. Biol. 1, 854-864 (1981); R. J. Kaufmann and P. A. Sharp, “Amplification And Expression Of Sequences Cotransfected with A Modular Dihydrofolate Reductase Complementary DNA Gene,” J. Mol. Biol. 159, 601-621 (1982); R. J: Kaufmann and P. A. Sharp, Mol. Cell. Biol. 159, 601-664 (1982); S. I. Scahill et al, “Expression And Characterization Of The Product Of A Human hnmune Interferon DNA Gene In Chinese Hamster Ovary Cells,” Proc. Natl. Acad. Sci. USA 80, 4654-4659 (1983); G Urlaub and L. A. Chasin, Proc. Natl. Acad. Sci. USA 77, 4216-4220, (1980).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Vectors containing the HIF-1 encoding DNA and control signals are inserted into a host cell for expression of HIF. Some useful expression host cells include well-known prokaryotic and eukaryotic cells. Some suitable prokaryotic hosts include, for example, E. coli, such as E. coli SG-936, E. coli HB101, E. coli W3110, E. coli X1776, E. coli X2282, E. coli DHI, and E. coli MRCI, Pseudomonas, Bacillus, such as Bacillus subtilis, and Streptomyces. Suitable eukaryotic cells include yeast and fingi, insect, animal cells, such as COS cells and CHO cells, human cells and plant cells in tissue culture.

The HIF-1α subunit protein may be any human or other mammalian protein, or fragment thereof. A number of HIFα subunit proteins have been cloned. These include HIF-1α, the sequence of which is available as GenBank accession number U22431, HIF-2α, available as GenBank accession number U81984 and HIF-3α available as GenBank accession numbers AC007193 and AC079154. These are all human HIFα subunit proteins. HIFα subunit proteins from other species, including murine HIF-1α (accession numbers AF003695, US9496 and X95580), rat HIF-1α (accession number Y09507), murine HIF-2α (accession numbers U81983 and D89787) and murine HIF-3α (accession number AF060194). Other mammalian, vertebrate, invertebrate or fungal homologues may be obtained by techniques similar to those described above for obtaining VHL homologues.

Variants of the HIFα subunits may be used, such as synthetic variants which have at least 45% amino acid identity to a naturally occurring HIFα subunit particularly a human HIFα subunit), preferably at least 50%, 60%, 70%, 80%, 90%, 95% or 98% identity.

Fragments of the HIFα subunit protein and its variants may be used. Such fragments are desirably at least 20, preferably at least 40, 50, 75, 100, 200, 250 or 400 amino acids in size. Alternately, such fragments may be 12 to 14 amino acids in size, or as small as four amino acids. Most desirably such fragments include the region 555-575 found in human HIF-1α or its equivalent regions in other HIFα subunit proteins. Optionally the fragments also include one or more domains of the protein responsible for transactivation.

The percentage homology (also referred to as identity) of DNA and amino acid sequences can be calculated using commercially available algorithms. The following programs provided by the National Center for Biotechnology Information) may be used to determine homologies: BLAST, gapped BLAST and PSI-BLAST, which may be used with default parameters. The algorithm GAP (Genetics Computer Group, Madison, Wis.) uses the Needleman and Wunsch algorithm to align two complete sequences that maximizes the number of matches and minimizes the number of gaps. Generally, the default parameters are used, with a gap creation penalty=12 and gap extension penalty—4. Use of either of the terms “homology” and “homologous” herein does not imply any necessary evolutionary relationship between compared sequences, in keeping for example with standard use of terms such as “homologous recombination” which merely requires that two nucleotide sequences re sufficiently similar to recombine under the appropriate conditions.

### Antibodies for Isolation of Stein Cells.

The antibodies are preferably monoclonal. Monoclonal antibodies may be produced by methods known in the art. These methods include the immunological method described by Kohler and Milstein in Nature, 495-497 (1975) and Campbell in “Monoclonal Antibody Technology, The Production and Characterization of Rodent and Human Hybridomas” in Burdon et al., Eds, Laboratory Techniques in Biochemistry and Molecular Biology, Volume 13, Elsevier Science Publishers, Amsterdam (1985); as well as by the recombinant DNA method described by Huse et al., Science, 246, 1275-1281 (1989).

In order to produce monoclonal antibodies, a host mammal is inoculated with a peptide or peptide fragment as described above, and then boosted. Spleens are collected from inoculated mammals a few days after the final boost. Cell suspensions from the spleens are fused with a tumor cell in accordance with the general method described by Kohler and Milstein in Nature, 256, 495-497 (1975). See also Campbell, “Monoclonal Antibody Technology, The Production and Characterization of Rodent and Human Hybridomas” in Burdon et al., Eds, Laboratory Techniques in Biochemistry and Molecular Biology, Volume 13, Elsevier Science Publishers, Amsterdam (1985). In order to be useful, a peptide fragment must contain sufficient amino acid residues to define the epitope of the molecule being detected.

If the fragment is too short to be immunogenic, it may be conjugated to a carrier molecule. Some suitable carrier molecules include keyhole limpet hemocyanin and bovine serum albumen. Conjugation may be carried out by methods known in the art. One such method is to combine a cysteine residue of the fragment with a cysteine residue on the carrier molecule. Other antibodies useful in the invention are commercially available. For example, antibodies against the CD34 marker are available from Biodesign of Kennebunk, Me.

The molecule may also be a fragment of an antibody. The fragment may be produced by cleaving a whole antibody, or by expressing DNA that encodes the fragment. Fragments of antibodies may be prepared by methods described by Lamoyi et al, Journal of Immunological Methods, 56:235-243 (1983) and by Parham et al., Journal of Immunology, 131:2895-2902 (1983).

Fragments of antibodies useful in the invention have the same binding characteristics as, or that have binding characteristics comparable to, those of the whole antibody. Such fragments may contain one or both Fab fragments or the F(ab′)₂ fragment. Preferably the antibody fragments contain all six complementarity determining regions of the whole antibody, although fragments containing fewer than all of such regions, such as three, four or five CDRs, may also be functional. The molecule is preferably labeled with a group that facilitates identification and/or separation of complexes containing the molecule.

### Labeling of Probes

The molecules that bind to antigens that are characteristic of stem cells, as described above, may be labeled in order to facilitate the identification and isolation of the stem cells. The label may be added to the molecule in accordance with methods known in the art. The label may be a radioactive atom, an enzyme, or a chromophore moiety. Methods for labeling antibodies have been described, for example, by Hunter and Greenwood, Nature, 144:945 (1962) and by David et al., Biochemistry 13:1014-1021 (1974). Additional methods for labeling antibodies have been described in U.S. Pat. Nos. 3,940,475 and 3,645,090. Methods for labeling oligonucleotide probes have been described, for example, by Leary et al., Proc. Natl. Acad. Sci. USA (1983) 80:4045; Renz and Kurz, Nucl. Acids Res. (1984) 12:3435; Richardson and Gumport, Nucl. Acids Res. (1983) 11:6167; Smith et al., Nucl. Acids Res. (1985) 13:2399; and Meinkoth and Wahl, Anal. Biochem. (1984) 138:267.

The label may be radioactive. Some examples of useful radioactive labels include ³²P, ¹²⁵I, ¹³¹I, and ³H. Use of radioactive labels have been described in U.K. 2,034,323, U.S. Pat. No. 4,358,535, and U.S. Pat. No. 4,302,204.

Some examples of non-radioactive labels include enzymes, chromophores, atoms and molecules detectable by electron microscopy, and metal ions detectable by their magnetic properties.

Some useful enzymatic labels include enzymes that cause a detectable change in a substrate. Some useful enzymes and their substrates include, for example, horseradish peroxidase (pyrogallol and o-phenylenediamine), β-galactosidase (fluorescein β-D-galactopyranoside), and alkaline phosphatase (5-bromo-4-chloro-3-indolyl phosphate/nitro blue tetrazolium). The use of enzymatic labels have been described in U.K. 2,019,404, EP 63,879, and by Rotman, Proc. Natl. Acad. Sci. USA, 47, 1981-1991 (1961).

Useful chromophores include, for example, fluorescent, chemiluminescent, and bioluminescent molecules, as well as dyes. Some specific chromophores useful in the present invention include, for example, fluorescein, rhodamine, Texas red, phycoerythrin, umbelliferone, luminol.

The labels may be conjugated to the antibody or nucleotide probe by methods that are well known in the art. The labels may be directly attached through a functional group on the probe. The probe either contains or can be caused to contain such a functional group. Some examples of suitable functional groups include, for example, amino, carboxyl, sulfhydryl, maleimide, isocyanate, isothiocyanate. Alternatively, labels such as enzymes and chromophores may be conjugated to the antibodies or nucleotides by means of coupling agents, such as dialdehydes, carbodiimides, dimaleimides, and the like.

The label may also be conjugated to the probe by means of a ligand attached to the probe by a method described above and a receptor for that ligand attached to the label. Any of the known ligand-receptor combinations is suitable. Some suitable ligand-receptor pairs include, for example, biotin-avidin or biotin-streptavidin, and antibody-antigen.

### Pharmaceutical Compositions

The pharmaceutical compositions of the invention comprise the compounds that modulate nitric oxide pathway combined with a pharmaceutically acceptable carrier. The term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and flngi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

### Materials and Methods

Six week old C57/BL6 recipient mice and transgenic GFP donor mice were purchased from Jackson laboratories (Bar Harbor, Me.). GFP mice were bred to homozygosity on the C57/BL6 background. These studies were approved by the University of Florida Institutional Animal Care and Use Committee.

### Hematopoietic Stem Cell Enrichment:

Bone marrow from 6 to 10 GFP animals were hemolyzed using ammonium chloride. Cells were incubated with a cocktail of rat anti-mouse antibodies to mature hematopoictic lineages as previously described. (Spangrude, G. J., Brooks, D. M. & Tumas, D. B. Blood 85, 1006-16 (1995)). Cells were washed and incubated with goat anti-rat magnetic beads. Lineage positive cells were removed with a magnet. The Linneg cells were incubated with an allophyocyanine conjugate of c-kit as well as a phycoerythrin conjugate of stem cell antigen-1. SKL cells were isolated using a fluorescence activated cell sorter. Purity was confirmed to be >90% by reanalysis.

### Transplants:

Adult bone marrow cells were flushed from the femora and tibiae of 2-4 month old GFP mice. Donor cells were hemolyzed and counted. Two to four month-old recipient animals were lethally irradiated with 950 cGy from a cesium¹³⁷ source. 2.0×10⁶ cells were infused retroorbitally under isofluorane anesthesia. Alternatively, 2×10³ SKL cells, or single SKL cells mixed with 2×10⁵ rescue marrow cells depleted of Sca-1^{pos} cells by Miltenyi beads, were transplanted. Equal numbers of irradiated control animals were set up.

### Single Cell Transplant:

SKL cells were suspended in a single drop of PBS, and single cells were identified using a dissecting microscope. A single SKL cell using a micropipette was removed and deposited in a microcentrifuge tube containing 100,000 C57/B6 Sca-1 depleted bone marrow cells. Single cell engrafted animals were identified by the presence of circulating GFP^{pos} hematopoietic cells. The animals were phlebotomized and then euthanized, and bones and bone marrow were isolated as described above for histological and FACS analysis. Immunohistochemistry:

The following monoclonal antibodies were used for the studies: primary antibodies included rat anti-CD31 (Santa Cruz), rabbit anti-VWF (DAKO), rabbit anti-HIF-1 (Novus), goat anti-SDF-1 (Santa Cruz), rabbit anti-GFP (Abcatn). Fluorescent antibody detection was by Alexa Fluor secondary antibodies (MolecularProbes) with wavelengths of 488 and 594. Slides were mounted with Vectashield containing DAPI (Vector).

### Fluorescent In Situ Hybridization:

X and Y chromosomes were labeled using STAR FISH whole chromosome paint probes (Cambio). Deparaffinized sections were sequentially retrieved and digested before overnight hybridization in a Hybrite oven (Vysis). For dual FISH/immunohistochemistry, ABC kits were used following the manufacturer's instructions with Vector Red used as the chromagen (Vector).

### Uptake of DiI-ac-LDL by Donor-Derived Endothelial Cells:

Transplant recipients received 20 μg of DiI-acLDL (1,1′-dioctadecyl-1,3,3,3′,3′-tetramethylindocarbocyamine labeled acetylated low-density lipoprotein) (Biomedical Technologies). Four hours after retroorbital injection, bones were placed in NBF for 20 hours, embedded in OCT, and sectioned. Marrow was carefully removed using a dissecting microscope and saturated with 8% sucrose in PBS. Alternatively, CD31^{pos} CD45^{neg} GFP positive cells were sorted one month post-transplant from a mouse transplanted with SKL cells, and sorted cells were treated for four hours with DiI-acLDL in ˜MEM medium. Uptake was measured by FACS analysis.

### Quantification of Donor-Derived Endothelial Cells by Flow Cytometry:

Flushed bone marrow was incubated from transplanted animals with anti-CD31 PE, rat anti-mouse CD 45 (Ly-5) APC, and propidium iodide. Donor-derived endothelial cells were identified based on expression of CD31, low expression of CD45, and green fluorescence. These cells endocytosed DiI-acLDL and were CD11b negative.

### Analysis of DNA Ccontent:

Donor-derived endothelial cells were sorted based on CD31 expression and low to negative expression of CD45. These cells were fixed with 50% ethanol and labeled them with 20 microgram/mL propidium iodide. Cell ploidy was analyzed using FACS. Disaggregated lymph node cells were used as a control.

### Fluorescent Microscopy and Confocal Analysis:

Images were capture on an Olympus BX 51 microscope equipped with and Optronics Magnafire digital camera system. Confocal imaging was performed on a Leica TCS SP2 AOBS Spectral Confocal Microscope.

### Measurement of Hypoxia:

Relative levels of hypoxia were measured using the Hypoxyprobe kit. Irradiated animals received an injection of pimonidazole 30 minutes prior to sacrifice at various time points following irradiation. Bones were collected as described above. Flushed bone marrow was analyzed by flow cytometry using fluorescein isothiocyanate conjugated anti-pimonidazole antibody provided in the kit.

Semi-Quantitative RT-PCR: Expression levels of hypoxia inducible factor-1, stromal derived factor-1, and HPRT by RT-PCR were measured. RNA was extracted using Trizol (Invitrogen) from irradiated animals on day 1, 3, 5, 7, 9, 11, and 13. mRNA was reverse transcribed using AMV reverse transcriptase to generate cDNA. PCR was performed using standard methods. Depicted gel was representative of 3 irradiated animals per time point.

### Measuring Protein Expression:

Protein expression of SDF-1 from a single femur flushed with PBS plus a protease inhibitor. 1% NP40 was added to the cell suspension. Cells were lysed by freeze/thaw, and cell debris was centrifuged and discarded. The supernatant was assayed using an ELISA kit for SDF-1. (R&D Systems).

### Transplants:

Two to four month-old female recipient animals were lethally irradiated with 950 cGy from a Cesium¹³⁷ source. We injected 2×10³ SKL cells into the retro-orbital sinus, or single SKL cells mixed with 2×10⁵ rescue marrow cells.

### Scoring Donor-Derived Endothelial Cells:

Sinusoidal capillary endothelial cells were defined by their characteristic morphology and location surrounding an irregular, endothelial lined space filled with erythrocytes. To be identified as a sinusoidal endothelial cell, the cell needed to be continuous with the endothelial surface of the sinusoid and have the characteristic elongated nucleus. Sections were then stained with MECA-32 specific antibody and a secondary antibody conjugated to DAB. DAB deposits a brownish insoluble residue to mark positive cells. Cover slips were then lifted, DNA was denatured, and hybridized with X and Y paint probes, and serial high power fields were again photographed at 1000×. Sinusoidal endothelial cells present on photographs from the H&E sections were located on the photographs from the FISH probed sections, and scored based on the presence of X and Y chromosomes. Denaturing the DNA caused changes in nuclear shape, and the movement of some erythrocytes. Only sections where the identity of cells could be clearly matched were used for the analysis. Cells containing XY or a single Y were scored as male, cells containing XX or a single X as female and cells with no X or Y were excluded from analysis.

### Example 1

### Radiation Induces Endothelial Damage

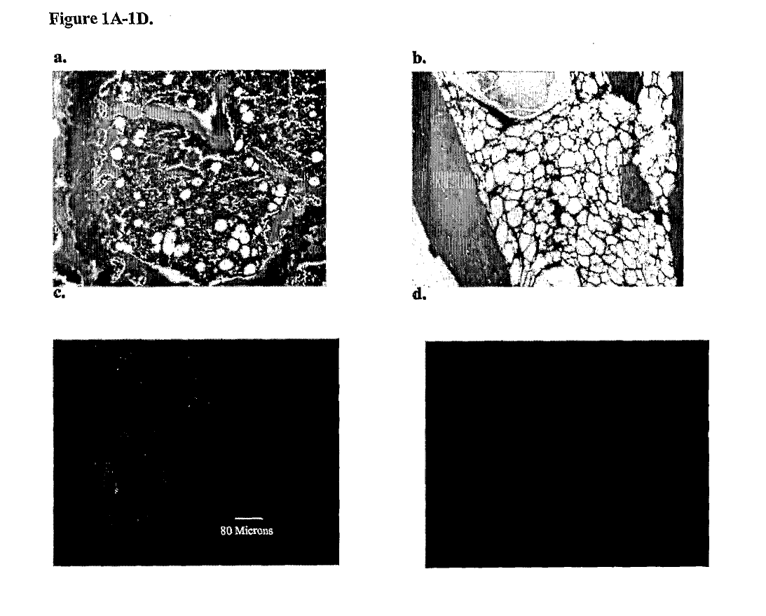

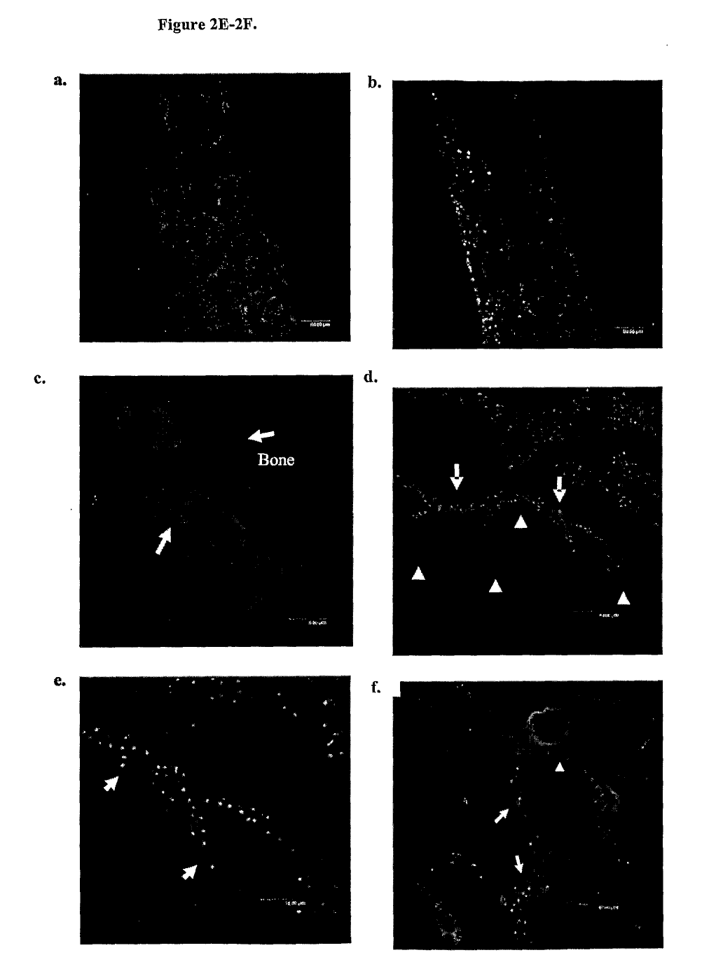

Characteristic changes in vascular endothelium in irradiated control and transplanted animals following lethal irradiation were observed. Hematoxylin and eosin stained sections of femora demonstrated increased diameter of the sinusoids during the first fourteen days following transplant. (FIGS. 1A and 1B). Endothelial damage was mediated in part by apoptosis. Using immunohistochemical staining with anti-caspase-3 antibody, upregulation of caspase-3, a mediator of the end stages of apoptosis, was observed in the endothelial and hematopoietic cells as early as three days and peaking seven days post-irradiation (FIG. 1C), in contrast to a healthy bone marrow that had not been exposed to radiation. FIG. 1D).

### Example 2

### Donor-Derived Cells are Dependent on Dying Endothelium for Repair Scaffold

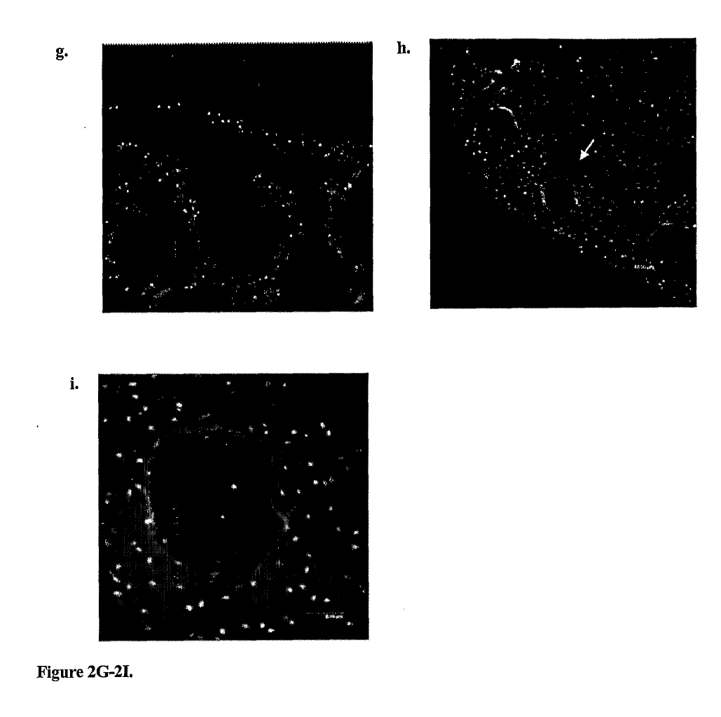

Donor-derived cells from whole bone marrow and sorted Sca-1^{pos} c-kit^{pos} Lin^{neg} (SKL) cells repaired the vascular niche following transplant. Green fluorescent protein (GFP) expression or the presence of a Y chromosome was used to demonstrate donor-derived cells. Engraftment first occurred along the endosteal surface (FIGS. 2A and 2B). Clusters of donor-derived cells were observed as early as five days post-transplant in the blood vessel immediately adjacent to the endosteal surface (FIG. 2C). By 8 days post-transplant, donor-derived cells lined the endosteal surface in a layer that in many places was one cell thick. Donor cells also formed a tube immediately adjacent to the endosteal surface along the remnants of a blood vessel (FIG. 2D). The cells predominantly contained a single X and single Y chromosome, making fusion unlikely (FIG. 2E). We also saw cells that streamed from the endosteal surface toward the center of the marrow space in narrow columns along regressing blood vessels. (FIGS. 2F and 2C). By one month post-transplant, donor cells had predominantly replaced host sinusoidal endothelial cells (FIGS. 2H and 2I). A total of 60 sinusoidal cross sections containing at least some endothelial cells from animals receiving SKL cells or whole bone marrow were scored for the presence of the Y-chromosome or, the presence of two Xs. The few cells that lacked a Y chromosome contained only one X, suggesting that these cells were sectioned through the nucleus. No differences between the repair activity of 2×10⁶ whole bone marrow or 2000 SKL cells one month post-transplant were observed.

### Example 3

### Cells Lining Tubes are Functional Endothelium

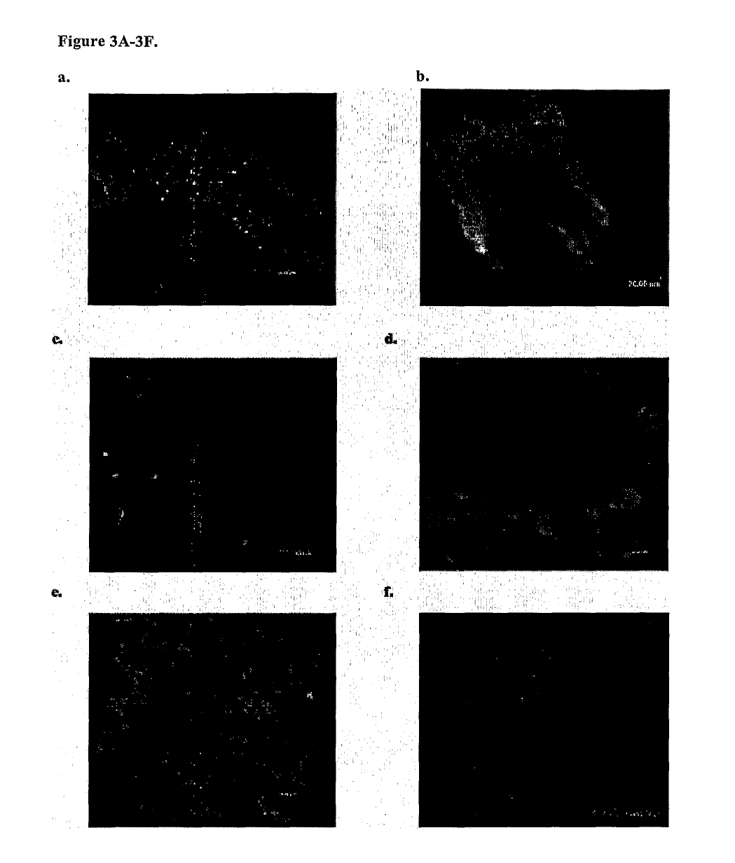

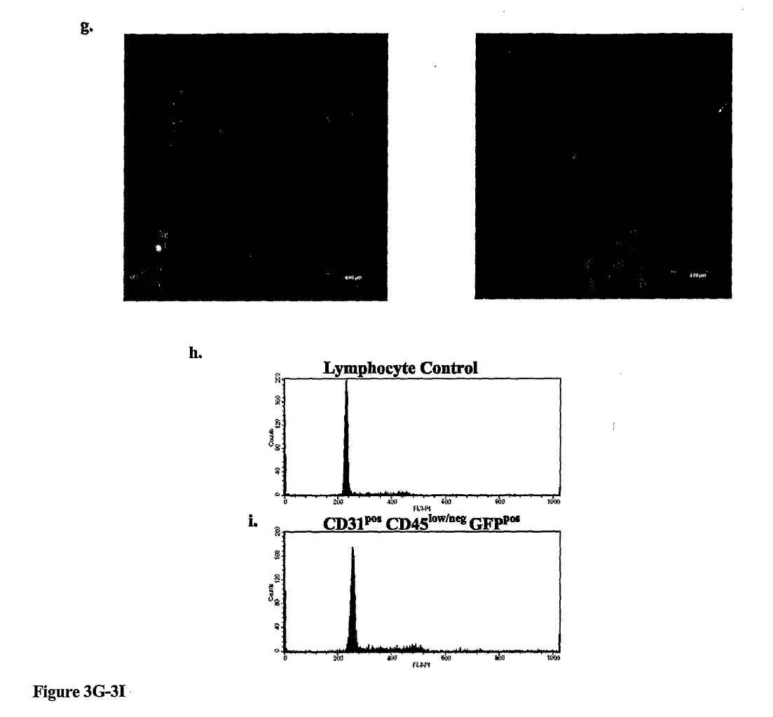

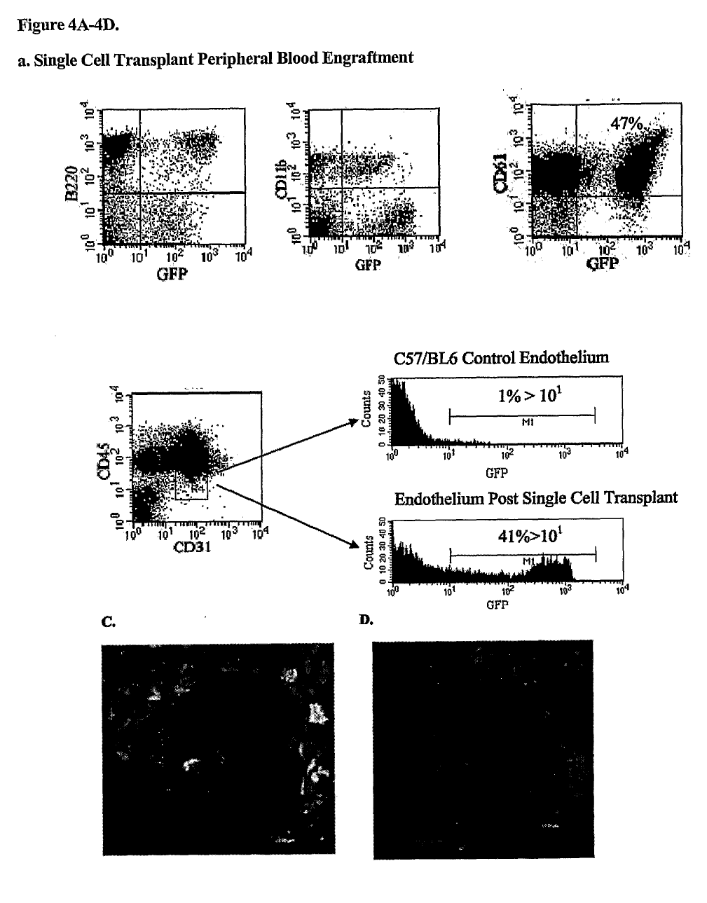

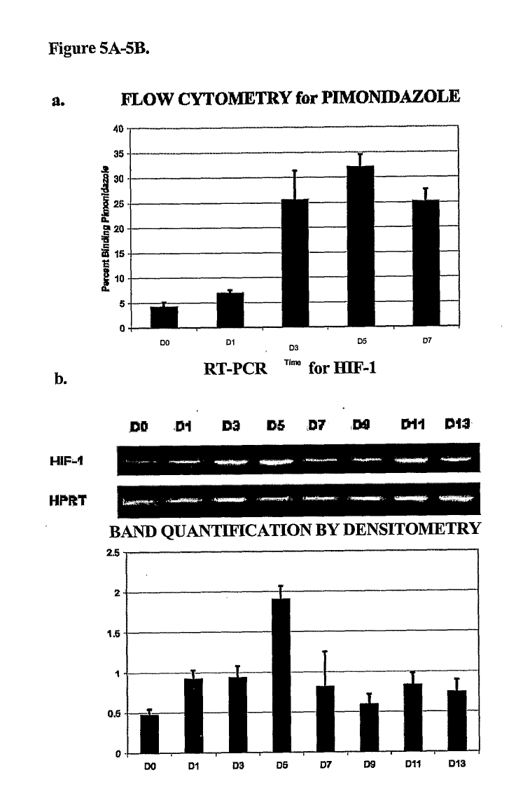

To determine whether these donor-derived cells were vascular endothelium, cells were stained for GFP or Y chromosome FISH to identify donor cells, combined with staining for platelet-endothelial cell adhesion molecule CD31, expressed by endothelial cells as well as erythrocyte progenitors and hematopoietic stem cells in the bone marrow, or von Willebrand factor (VWF), specific to endothelial cells, megakaryocytes and platelets. Donor derived cells were observed that expressed CD31 (FIGS. 3A and 3B) and VWF (FIG. 3C) as early as one week post-transplant at the edges of developing tubes. Similar cells were observed three months following secondary transplants from primary transplants involving 2000 SKL cells (FIG. 3D). Finally, to determine whether these cells functioned as blood vessel endothelium, animals that had been transplanted with 2000 SKL cells, were injected with DiI-acLDL one month post-transplant. DiI-acLDL is endocytosed by healthy endothelial cells.

FIG. 3E shows the network of functional bone marrow sinusoids have endocytosed DiI-acLDL (red) four hours following injection in a healthy animal. FIGS. 3F and 3G demonstrate donor-derived endothelial cells expressing GFP that have also endocytosed DiI-acLDL. The DNA content of GFP positive CD31^{pos} CD45^{low/neg} cells was measured by flow cytometry using propidium iodide staining. These cells were predominantly diploid, confirming that the process of vasculogenesis post-transplant is not due to fusion (FIGS. 3H and 3I).

### Example 4

### Endothelial Repair can be Mediated by Single SKL Hematopoietic Stem Cells (Hemangioblasts)

To determine whether hematopoietic stem cells function as hemangioblasts in the repair of bone marrow sinusoids following transplant, single cell transplants were performed using double-sorted GFP-positive male SKL cells. Three single cell engrafted animals were identified based on the presence of GFP positive circulating hematopoietic cells seven to nine months post-transplant. Circulating donor-derived lymphocytes, granulocytes and platelets were observed by flow cytometry (FIG. 4A). Following single-cell transplant, the percentage of circulating donor-derived hematopoietic cells ranged from about 20-80% in the three engrafted recipients. Flow cytometry was also used to identify a subset of bone marrow cells that endocytosed Dil-acLDL, expressed CD31, and were CD45^{low} and CD11b^{neg}, a subset that contains sinusoidal endothelium. Flow cytometry showed expression of GFP in this population of cells at levels that was similar to the level of hematopoictic engraftment within each animal (19-40%). (FIGS. 4B-4D). GFP^{pos} endothelial cells were seen lining sinusoids in the bone marrow (FIGS. 4E-4F).

### Example 5

### Endothelial Repair is Preceded by Marrow Hypoxia, Upregulation of Hypoxia-Inducible-Factor-1α, and Increased Expression of Stromal Derived Factor-1

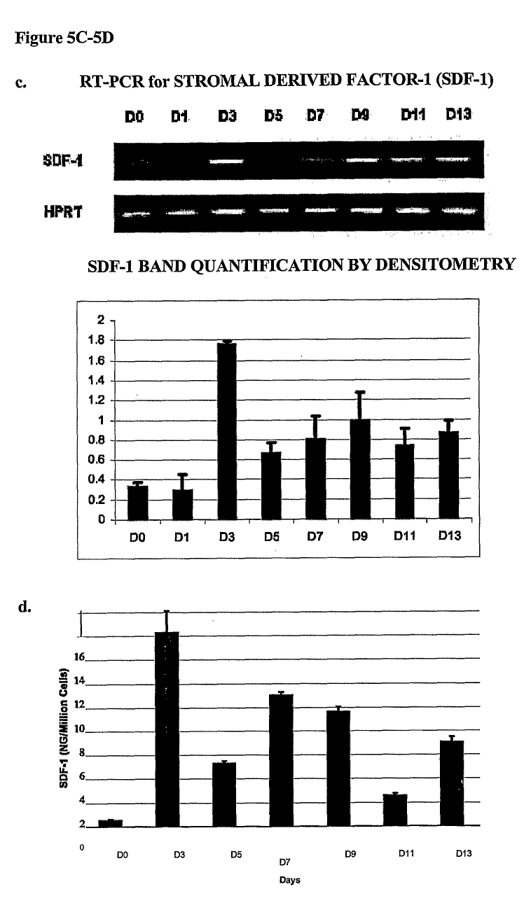

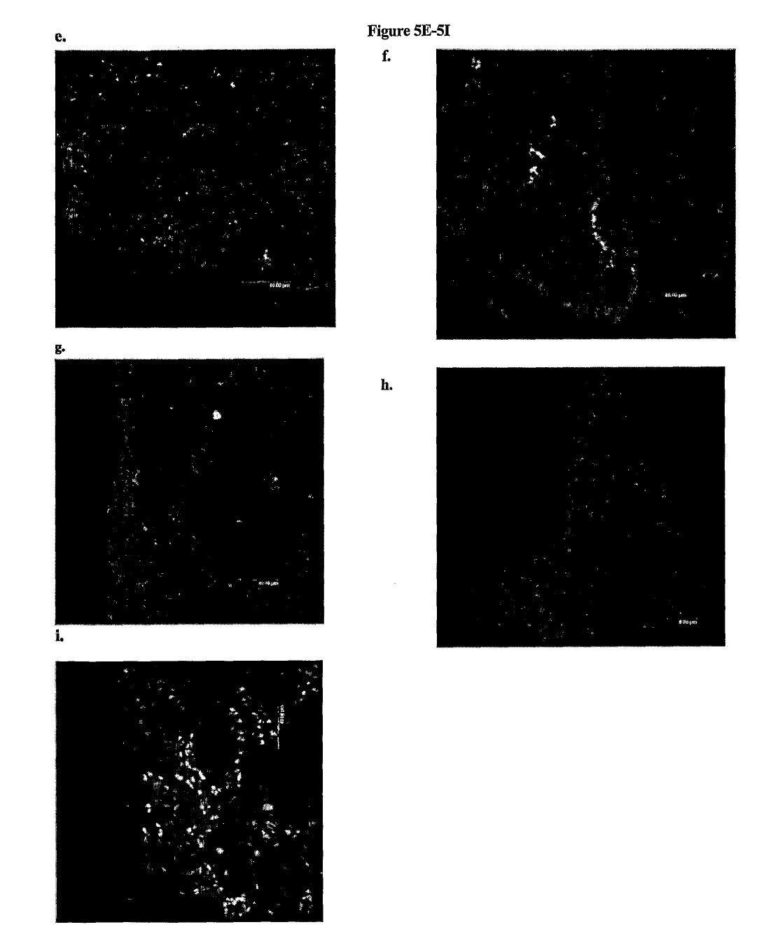

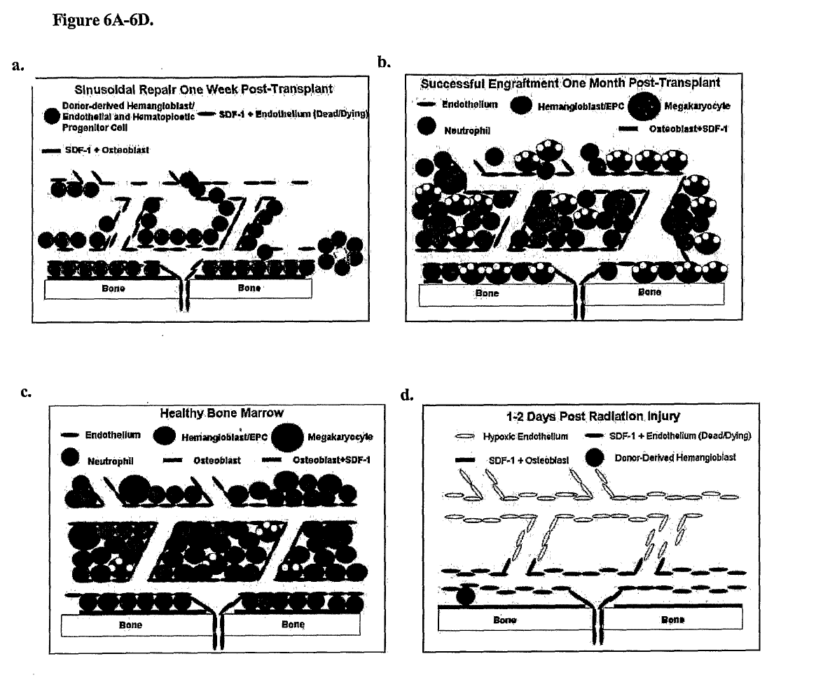

Since vasculogenesis is typically driven by hypoxia, the compound, pimonidazole, that binds to tissues based on oxygen tension was used to determine whether the bone marrow becomes more hypoxic following lethal irradiation. The percentage of cells that bind pimonidazole demonstrated increases of eight-fold by five days after irradiation (FIG. 5A). Expression levels of the alpha subunit of hypoxia inducible factor-1 (HIF-1α) by semi-quantitative RT-PCR were then measured. Transcriptional upregulation of this subunit as well as post-translational modifications of HIF-1 are two major ways cells respond to hypoxia. HEF-1α expression increased 4-fold five days post-irradiation (FIG. 5B).

HIF-1 regulates numerous proteins involved with angiogenesis, including stromal derived factor-1 (SDF-1). SDF-1 regulates the migration, survival and proliferation of hematopoietic stem cells and endothelial progenitors. The expression of SDF-1 was measured by RT-PCR, ELISA, and immunohistochemistry. SDF-1 transcript levels peaked three days post-transplant (FIG. 5C). Per-cell SDF-1 protein expression increased 16-fold by three days post-transplant as measured by ELISA (FIG. 5D). HIF-1α (green) and SDF-1 (red) protein co-localize first in the osteocytes, along the endosteal surface and the peri-endosteal blood vessels three days post-irradiation (FIGS. 5E-5H). Co-expression of HIF-1 and SDF-1 moves toward the center of the marrow space five days post-irradiation (FIG. 5I). This pattern coincides precisely with the migration pattern of the engrafting cells.

FIGS. 7A and 7B is a scan of a cyto stain showing sections from a female mouse transplanted with male bone marrow. The sections are of the bone marrow after transplant. The dark brown staining is for an endothelial specific marker—MECA 32 that stains new endothelium. The green dots are Y chromosome showing male cells, the red dots are the X chromosome. Essentially all of the new sinusoids are being made by male Y positive cells in this area of the bone. Supports the claim that the donor HSC stem cells are first required to rebuild the vascular niche before a successful engraftment can occur. Thus adult hemangioblast activity can be used to improve Bone marrow transplant engraftment rates and outcome.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

All references cited herein, are incorporated herein by reference.

1. A composition comprising a stem cell transduced with an expression vector encoding at least one angiogenic factor wherein the stem cell is derived from bone marrow, and is Lin⁻, Sca-1⁺, c-kit⁺ or Lin⁻ Sca-1⁺ c-kit⁺; wherein the angiogenic factor is hypoxia inducible factor-1; and, the vector is a plasmid or viral vector.

2-7. (canceled)

8. The composition of claim 1, wherein the hypoxia inducible factor-1 is secreted.

9-10. (canceled)

11. The composition of claim 1, wherein the stem cell is optionally cultured with a compound which modulates nitric oxide pathways.

12. The composition of claim 11, wherein the drug is selected from any one of sildenafil (Viagra®), vardenafil (Levitra®), tadalafil (Clalis®), apolipoprotein-E, nitroglycerine, L-arginine, nitrate esters, isoamylynitrite, SIN-1, cysteine, dithiothreitol, N-acetylcysteine, mercaptosuccinic acid, thiosalicylic acid, and methylthiosalicylic acid.

13. A composition comprising a stem cell transduced with a vector expressing pro-angiogenic growth factors wherein the stem cell is derived from bone marrow, and is Lin⁻, Sca-1⁺, c-kit⁺ or Lin⁻ Sca-1⁺ c-kit⁺; wherein the pro-angiogenic growth factors are selected from the group consisting of SDF-1, HGF, IL-8, PGF, TGF and TNF.

14-18. (canceled)

19. The composition of claim 13, wherein the pro-angiogenic factors are secreted extracellularly.

20. An isolated or purified population of mammalian hematopoietic stem cells comprising at least one stem cell marker and wherein the purified population of mammalian hematopoietic stem cells are substantially free of cells that do not express the stem cell marker; and, the hematopoietic stem cells are Lin⁻ Sca⁺ c-kit⁺ and are derived from postnatal mammals wherein the hematopoietic stem cells are Lin⁻ Sca⁺ CD34⁺, and are human hematopoietic stem cells.

24. The isolated or purified population of mammalian hematopoietic stem cells of claim 20, wherein the stem cells are treated with compounds that modulate the nitric oxide production pathway.

25. The isolated or purified population of mammalian hematopoietic stem cells of claim 20, wherein the compound is selected from at least one compound identified by sildenafil (Viagra®), vardenafil (Levitra®), tadalafil (Clalis®), apolipoprotein-E, nitroglycerine, L-argenine, nitrate esters, isoamylynitrite, SIN-1, cysteine, dithiothreitol, N-acetylcysteine, mercaptosuccinic acid, thiosalicylic acid, and methylthiosalicylic acid.

26. The isolated or purified population of mammalian hematopoietic stem cells of claim 20, wherein the purified population of mammalian hematopoietic stem cells that are Lin⁻ Sca⁺ constitute 15-100% of the total population.

27. The isolated or purified population of mammalian hematopoietic stem cells of claim 20, wherein the purified population of mammalian endothelial stem cells that are Lin⁻ Sca⁺ CD34⁺ constitute 15-100% of the total population.

28. A method of reconstituting hematopoiesis and/or repair of vasculature in a patient comprising administering to a patient a stem cell identified by marker phenotype of Lin⁻ Sca⁺ c-kit⁺ up to 2×10⁶ Lin⁻ Sca⁺ c-kit⁺ stem cells, wherein the stem cells are administered with one or more compounds that modulate the nitric oxide pathway and the at least one compound that modulates the nitric oxide pathway is selected from sildenafil (Viagra®) vardenafil (Levitra®) tadalafil (Clalis®), apolipoprotein-E, nitroglycerine, L-arginine, nitrate esters, isoamylynitrite, SIN-1, cysteine, dithiothreitol, N-acetylcysteine, mercaptosuccinic acid, thiosalicylic acid, and methylthiosalicylic acid.

33. The method of claim 31, wherein the compounds that modulate the nitric oxide pathway are administered prior to, in conjunction with, and/or post administration of the stem cells.

34. The method of claim 31 wherein reconstituted cells are CD31^{pos} CD45^{low/neg} cells.
